(19) United States
(12) Patent Application Publication
(10) Pub. No.: US 2008/0086142 A1
(43) Pub. Date: Apr. 10, 2008

(54) Products and Methods for Delivery of Material to Bone and Other Internal Body Parts

(76) Inventors: Andrew Christopher Kohm, Burlingame, CA (US); Hugues Malandain, Mountain View, CA (US); Lex Philip Jansen, Pleasanton, CA (US)

Correspondence Address: KILPATRICK STOCKTON LLP - 55461, 1001 WEST FOURTH STREET, WINSTON-SALEM, NC, 27101, US

(21) Appl. No.: 11/862,857

(22) Filed: Sep. 27, 2007

Related U.S. Application Data
(60) Provisional application No. 60/849,860, filed on Oct. 6, 2006.

(51) Int. Cl. A61B 17/58

(52) U.S. Cl. 606/92; 606/93; 606/94

(57) ABSTRACT

Disclosed are products and methods for delivery of a material to an internal body part such as bone. In one variation, the product includes an access member having a side port aperture positioned near the distal end of the access member for extruding a material to a body part within a subject, wherein the access member comprises a variation in the internal diameter adjacent to the side port aperture, and wherein the variation in the internal volume adjacent to the side port aperture reduces the tendency of the material extruded from the side port to adhere to the material remaining in the access member.

### PRIORITY CLAIM TO RELATED APPLICATIONS

The present invention claims priority under 35 U.S.C. § 119(e) to U.S. Provisional Patent Application Ser. No. 60/849,860, filed Oct. 6, 2006. The disclosure of U.S. Provisional Patent Application Ser. No. 60/849,860 is hereby incorporated by reference in its entirety herein.

### FIELD OF INVENTION

The present invention relates to products and methods for delivery of a material to an internal body part such as bone.

### BACKGROUND

A variety of conditions may warrant repair and/or replacement of an internal body part, such as bone. For example, to repair a bone fracture, an adhesive agent may be applied to adhere sections of the separated bone together. Also, a bone filler material may applied to a bone to replace degenerated tissue and/or to provide a biologically compatible matrix to support or reinforce the bone.

In many instances, it can be preferred to minimize the invasiveness of a procedure used for treatment and repair of an internal body part, such as bone. Minimally invasive spine fracture surgeries may be performed by accessing the fractured bone using a cannula-based technology. For example, a cannula-based bone filler device may comprise an outer cannula and an inner rod-like tamping instrument. The cannula may be loaded with an aliquot of a bone repair material using an injection nozzle and syringe, and the repair material urged to the site requiring repair using the tamping instrument.

There may be significant difficulties in accessing the body part that needs to be repaired even when using a cannula based delivery system. Many cannula based delivery systems are designed such that the material to be emplaced in, or delivered to, an internal body part will be forced out of the cannula to exit from an aperture at the end of the cannula. However, there is often a need to be able have the material exit the delivery cannula from the side of the cannula. For example, in some cases, the body of the cannula may be used to prevent the extruded material from being delivered to a part of the bone where there may be a breach. Or, there may be difficulty in completely accessing the body parts without being able to impart some directionality to the delivery of the material. Using a cannula having a side port can, however, result in a portion of the distal end of the cannula becoming coated with the material being delivered to the body part as the material is extruded from the side of the cannula. In some cases the material being delivered comprises an adhesive component, or is highly cohesive in nature. For example, the material may comprise a cement that can harden (i.e., cure). Upon curing, the material may harden around the cannula and/or stick to the unspent material remaining in the cannula. For example, in some cases the material delivered may shrink around the cannula and/or expand into the body part leaving less room for the cannula. This can occur when there is a need to leave the cannula in place for several minutes during the procedure. In such instances, it can be difficult to extricate the cannula from the material being delivered to the bone or other body part, especially where there is a significant amount of the material left inside of the cannula that can bond to the material covering the outer surface of the cannula.

There may also be difficulties in using a cannula based delivery system where there is restricted access to the patient. For example, to optimize emplacement of the correct amount of a bone repair material into the bone, the repair material may utilize a radio-opaque tracer to allow for visualization of the repair material as it is being emplaced. The patient may then be positioned in an X-ray apparatus known as a C-arm, with the X-ray transmitter (one arm of the “C”) above the patient, and the receiver (the other arm of the “C”) underneath the patient. Optimal positioning of the C-arm (or other radio-imaging device) may require that the imaging device is positioned very close to the site that is being treated. For example, optimal positioning of the C-arm may require that the arms of the C-arm device be only a few inches from the patient's torso. Close positioning of the C-arm, however, may make it difficult for the physician to access the cannula, as for example, where a substantially straight tamping instrument must be inserted into the cannula to push the material being delivered to the bone through the cannula body.

Thus, there is a need to provide methods and products that can be used to deliver therapeutic materials to the interior of a patient's body, but that reduce the tendency for the device to become embedded in the therapeutic material once the material hardens in situ. There is also a need to provide methods and products that can be used to deliver therapeutic materials to the interior of a patient's body where access to the entry site for the cannula may be restricted. Such products may be provided in a form so as to be a self-contained unit, or may be provided as accessories for use with currently available cannula based delivery systems, such as those used to emplace bone filler and other materials into bones or other body parts.

### SUMMARY OF THE INVENTION

Embodiments of the present invention comprise products and methods for delivering material to a predetermined location in a subject, such as an internal body part or region. The present invention may be embodied in a variety of ways.

In certain embodiments, the present invention comprises methods for delivery of a material to a body part in a subject. The method may, in certain embodiments, use an access member configured to provide percutaneous surgical access to the body part, wherein at least a portion of the internal volume of the access member is filled with at least a portion of the material to be delivered to the body part, and wherein the access member comprises a side port aperture positioned near the distal end of the access member for extruding the material to the body part. Also, in an embodiment, the access member comprises a variation in the internal diameter adjacent to the side port aperture. The method may, in certain embodiments, comprise inserting the access member in the subject such that the distal end of the access member is positioned within the body part, or juxtaposed adjacent to an aperture in the body part, and urging an inner member at least partially through the access member to deliver at least a portion of the material to the body part. In an embodiment, the variation in the internal volume adjacent to the side port aperture reduces the tendency of the material extruded from the side port to adhere to the material remaining in the access member and/or for the access member to become embedded in the material that has been delivered to the body part.

In other embodiments, the present invention may comprise a product comprising an access member configured to provide percutaneous surgical access to a body part in a subject comprising a side port aperture positioned near the distal end of the access member for extruding a material to a body part within a subject, wherein the access member comprises a variation in the internal diameter adjacent to the side port aperture. In certain embodiments, the variation in the internal volume adjacent to the side port aperture reduces the tendency of the material extruded from the side port to adhere to the material remaining in the access member and/or for the access member to become embedded in the material that has been delivered to the body part.

Other embodiments and further details on various aspects of the present invention are set forth in the following description, figures, and claims. It is to be understood that the invention is not limited in its application to the details set forth in the following description, figures, and claims, but is capable of other embodiments and of being practiced or carried out in various ways.

### BRIEF DESCRIPTION OF THE FIGURES

FIG. 1 illustrates a prior art cannula based bone filler delivery system having a side port for extrusion of a material into a body part of interest.

FIG. 2 illustrates a prior art bone filler delivery system having a side port for extrusion of a material being used to emplace a bone filler material in a bone.

FIG. 3 illustrates a bone filler delivery system having a side port and an internal angled step being used to extrude a bone filler material in accordance with an embodiment of the present invention, where panel A shows the inner member at a first position, and panel B shows the inner member at a second, more distal position.

FIG. 4, panels A and B, illustrates a cross-sectional view of a bone filler access member having a side port and a straight ramp, where the straight ramp is a separate piece that may be inserted into the access member in accordance with an embodiment of the present invention.

FIG. 5 illustrates angled steps having surfaces that are flat (panel A), concave (panel B), or convex (panel C), simple concave (panel D), and a compound concave (panel E) in accordance with various embodiments of the present invention.

FIG. 6 illustrates different structures for insertion into an access member in accordance with alternate embodiments of the present invention including a flat angled step (panels A-C); a concave ramp having a squared-off end (panels D-F); multiple steps (panels G-I); a plurality of convex and concave surfaces comprising a wave-type shape (panels J-L); and a concave ramp having a tapered proximal end (panels M-O).

FIG. 7 illustrates different structures positioned within an access member having a side port for extrusion of a material into a body part of interest including an angled step (panel A), a plurality of convex and concave surfaces comprising a wave-type shape (panel B); a reverse angled step (panel C); and multiple steps (panel D), in accordance with various embodiments of the present invention. Panel E shows the use of an access member comprising multiple steps adjacent to the side port being used to extrude a material in accordance with an embodiment of the present invention.

FIG. 8, Panels A-C show an bifurcated ramp with a dividing fin, where panel A shows a perspective view, panel B shows the piece inserted in an access member being used to deliver a material to a body part, and panel C shows a view looking into the side port aperture of the piece inserted in an access member in accordance with various embodiments of the present invention.

FIG. 9 illustrates access members that have a tapered end comprising at least a portion of the distal end of the access member that includes the side port in accordance with alternate embodiments of the present invention where panel A shows an access member having a concave distal end as the back side of the side port aperture, and panel B shows an access member having a flat distal end as the back side of the side port aperture.

FIG. 10 illustrates top views of different shapes for a side port aperture including: a compound curve (panel A); a tear drop (panel B); a diamond (panel C); interlaced diamonds (panel D); an opening with a serrated edge (panel E); an eye-shaped opening (panel F); a bifurcated opening (panel G); and a serrated bifurcated opening (panel H) in accordance with various embodiments of the present invention.

FIG. 11 illustrates a handle having two side injection apertures and one central injection aperture that may be used with an access member and an inner member in accordance with an embodiment of the present invention where panel A shows a cross-sectional view and panel B shows a top-perspective view.

FIG. 12 illustrates a handle having multiple side injection ports (i.e., apertures) and one central port that may be used with an access member in accordance with alternate embodiments of the present invention, wherein panel A shows a top view, and panel B shows a perspective view of an inner member being inserted into the handle.

FIG. 13 illustrates use of an access member having a side injection aperture to emplace a material in a body part in accordance with an embodiment of the present invention.

FIG. 14, panels (A) and (B), illustrate two systems comprising products in accordance with alternate embodiments of the present invention FIG. 15, panels (A) and (B), illustrate alternate embodiments of a kit of the present invention.

FIG. 16 illustrates a method for using a bone filler device in accordance with an embodiment of the present invention.

### DETAILED DESCRIPTION

Embodiments of the present invention provide products and methods for delivering a material to a predetermined location in a subject, such as an internal body part or region. In certain embodiments, the products and methods of the present invention may reduce the tendency for a device being used to deliver a material to the interior of a patient's body to become embedded in the material once the material hardens in situ. In certain embodiments, the present invention may also provide access to internal body regions and/or internal body parts where there may be limited clearance to perform the delivery procedure. Particular embodiments of the present invention may comprise products and methods to emplace a bone filler or a bone cement or other material for the treatment and repair of bone.

As used herein, a subject is an animal. For example, the subject may comprise a mammal. In one embodiment, the subject may be a human. In certain embodiments, the subject is a patient seeking medical treatment (e.g., for repair of a bone or other body part). The user of the products, methods, and systems of the present invention may be a physician, veterinarian, or other type of health care professional. In another embodiment, however, a user of the product may be accessing a particular location in his or her own body, as for example, for periodic delivery of a therapeutic material.

As used herein, a predetermined location in a subject may comprise a body part, a region within the body, or a region within a body part. As used herein, an internal body part may comprise a bone or bones (e.g., a vertebral body or spinal disc). An internal body part may also comprise a cartilage, a tendon, muscle, a vein, an artery, or an organ (e.g., intestines, stomach, liver or lung), or part thereof, that may need to be accessed and repaired. Or, the predetermined location may comprise an internal body region, such as the vasculature, abdomen, or other body regions.

In certain embodiments, the body part may comprise a bone or a bone interior. For example, the predetermined location may comprise a portion of a spine, such as a vertebral body or a spinal disc. For example, due to various traumatic or pathologic conditions, such as osteoporosis, a vertebral body can experience a vertebral compression fracture (VCF). In such conditions, at least a part of the vertebral bone can be compacted, causing a decrease in height of the vertebra. In many cases, vertebral height is lost in the anterior region of the vertebral body. Thus, the products and methods of the present invention may by used to repair a vertebral body. The present invention is not, however, limited in application to vertebrae, and may be used to repair other parts of a living or non-living organism. For example, in embodiments, the products, methods, kits and systems of the present invention can be deployed in other bone or tissue types, such as in a vertebral disc, an arm bone, a leg bone, a knee joint, and the like.

As used herein, an access member comprises a device for accessing a predetermined location in a subject. The access member may have a distal end to be positioned in or near the body part to be treated, and a proximal end to be accessible to a user (e.g., physician). The inner volume of the access member may provide a path to access a region or a body part that is located within the subject's body. The access member may be any type of device that can extend from the location of interest (e.g., a bone or an organ) to be accessible to a user of the access member. For example, the access member may be designed to extend from an internal body part in a subject to outside of the subject's body. The access member may be an elongated hollow member such as a hollow cylinder or tube. In certain embodiments, the access member may comprise a cannula or catheter.

As used herein, an inner member is a device that fits within an access member. An inner member may be used to urge a material from the end of the access member that is outside of the body (e.g., the proximal end) towards the end of the access member that is inside the body (e.g., the distal end). The inner member may be an elongated cylinder that is closed off at the end (e.g., the distal end) that comes in contact with the material. The inner member may comprise a handle at one end, to allow a user to push the inner member through the access member. The inner member may be a rod, or a stylet, a plunger, or a tamping instrument. Or other types of inner members used in the art of cannula-based delivery systems may be used.

As used herein, the terms “side port” and “side port aperture” refer to an aperture that is positioned along the length of an access member rather than at the end of the access member. As used herein, the length of an access member defines the distance from the proximal end of the access member to the distal end of the access member and is perpendicular to the cross-section of the access member.

As used herein a ramp is a continuous, non-vertical incline from a first plane in space to a second plane in space. A step is a vertical incline from one plane in space to another plane in space. An angled step comprises a step having an incline as at least part of the horizontal surface of the step; a reverse angled step is a step having at least one non-vertical incline (e.g., less than 90 degrees) as at least part of the vertical surface. A wave comprises a surface with a plurality of convex and concave surfaces to for peaks and troughs such as series of waves.

As used herein, the term “reposition the access member” includes moving the access member in any manner and includes rotating, twisting, jiggling, pulling on (i.e., pulling the access member in a proximal direction) or otherwise moving the distal end of the access member in relation to the material that is delivered to the body part.

Also, as used herein, a material for emplacement within, or delivery to, a predetermined location in a subject may comprise any material that is biologically compatible with the predetermined location of interest. For example, in alternate embodiments, the material may comprise a bone filler material or an adhesive. The material may further comprise any one of a therapeutic drug, a tissue graft, a population of cells, a biological matrix, or any other physiological material for delivery to a location in a body. As used herein, a bone filler material comprises any material that may be used for the treatment of bone. A variety of materials have been described for use as bone filler materials (see e.g., U.S. Pat. Nos. 4,904,257, 6,203,574, 6,579,532, 6,740,093, and Patent Application No. 2005/0136038 for descriptions of bone filler materials, and each of which are incorporated herein by reference in its entirety for the description of the bone cement but not to limit the terms used herein). In one embodiment, the bone filler or treatment material may comprise an adhesive. For example, in an embodiment, the material for emplacement in the body part comprises a bone cement. Example bone cements that may be used include, but are not limited to polymethyl methacrylate (PMMA) based-bone cement, resorbable bone cement, bone cement that includes osteo-inductive materials, and bone cement with a relatively fast cure time (e.g., less than 25, 20, 15, 10, 5 or 3 minutes). Example bone cements that may be used include KYPHX® HV-R bone cement (dough time of 8 to 16 minutes; set time of about 19.8 to 21.3 min), and KYPHX® QV-R bone cement (dough time of about 4.5 minutes to 9 minutes; set time of about 12.9 to 13.4 minutes), commercially available from Kyphon, Inc. In an embodiment, an injectable calcium-phosphate cement (e.g., Weitao et al., J. Postgrad. Med., 2007, 53:34-38), hydroxyapatite composite materials (e.g., hydroxyapatite/ceramic composites and/or hydroxyapatite/polymer composites) may be used.

In addition, the words “proximal” and “distal” refer to directions closer to, and away from, respectively, an operator (e.g., surgeon, physician, nurse, technician, etc.) who would insert the access member of the present invention into the patient, with the tip-end (i.e., distal end) of the device inserted inside a patient's body. Thus, for example, the cannula end inserted inside the patient's body would be the distal end of the cannula, while the cannula end outside the patient's body would be the proximal end of the cannula.

Furthermore, in this specification and the appended claims, the singular forms “a,” “an” and “the” include plural referents unless the context clearly dictates otherwise. Thus, for example, the term “a lumen” is intended to mean a single lumen or a combination of lumens, “a fluid” is intended to mean one or more fluids, or a mixture thereof.

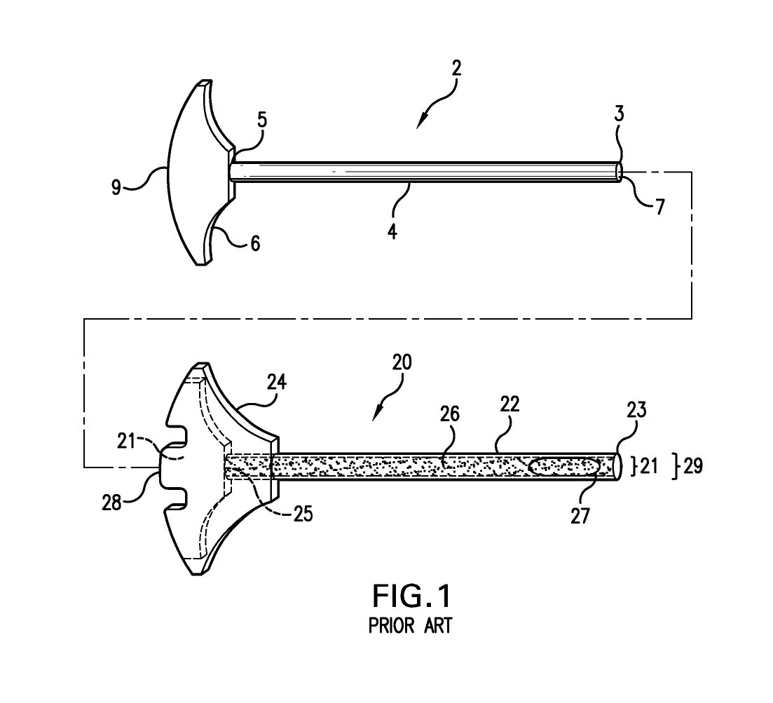

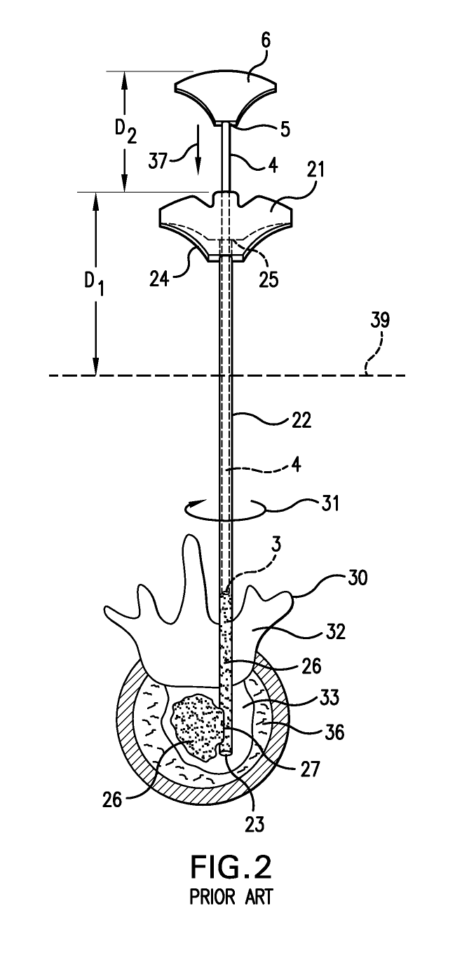

To better understand the present invention, a bone filler device of the prior art for delivery of bone filler material to a bone is shown in FIGS. 1 and 2. FIG. 1 shows the individual parts of the bone filler device, and FIG. 2 shows the device being used to emplace a bone filler material into a bone. Although a vertebral body is depicted in FIG. 2, it will be understood that the devices, methods and systems of the present invention may be used to deliver a material to a wide variety of body parts of interest.

Thus, as shown in FIG. 1, a bone filler device may comprise a first outer part 20, comprising a hollow cylinder or tube 22 and handle 24, and a second inner part 2, comprising a closed cylinder 4 and handle 6. The outer cylinder 22 comprises a distal end 23 (i.e., the end farthest from the user), and a proximal end 25 (i.e., the end closest to the user). Also, the inner cylinder 4 comprises a distal end 3 and a proximal end 5.

As illustrated in FIGS. 1 and 2, the two concentric cylinders 4, 22, may be used to emplace a paste-like bone filler material 26 into the bone. The open interior 21 of the outer cylinder 22 of the bone filler device is designed to provide access to the bone being repaired by providing a cannula-type access into which the inner cylinder 4 fits. The inner cylinder 4 generally has the distal end 3 closed off to form a substantially flat surface 7. To emplace the bone filler material, the outer cylinder 22 may be filled with the required bone filler material 26. Generally the outer cylinder will be loaded with enough bone filler material so that the cylinder comprises material from the distal end 23 to the proximal end 25. In some cases, however, the outer cylinder is only partially filled with the bone filler material. Also, in some cases, the filled outer cylinder may be inserted into the bone through a second cylindrical cannula (not shown) having a slightly larger inner diameter than the outer diameter 29 of the outer cylinder 22 of the bone filler device.

For some delivery systems, the distal end 23 of the outer cylinder 22 comprises the opening used to emplace the material 26 in the body part of interest (e.g., a bone). In some cases, however, a side port 27 may be used for directional delivery of the bone filler material (FIGS. 1 and 2). In this way, the user may deliver a bone filler material in a particular direction or directions. For example, a bone delivery system having a side port may be preferred where there is a breach in the bone that can be blocked using the body of the outer cylinder. Also, a side port may be preferred where it is difficult to access the entire body part using a unidirectional delivery system. Thus, a user may start the flow of the bone filler material in one direction, and then gently rotate or twist 31 the cannula system to allow for delivery of the bone filler material in a plurality of different directions (FIG. 2). In an embodiment, the bone filler material may be delivered in all directions such that a radius of 360 degrees is utilized.

FIG. 2 shows emplacement of a material 26 in a vetebral body 30. As shown in FIG. 2, the outer cylinder 22 may be inserted through the cortical bone 32 of the vetebra and into the cancellous bone 36. For example, in some cases a catheter and balloon may be used to compact bone and to create a internal void space 33 in a bone 36 requiring repair. Then, the outer cylinder 22 of the bone filler device may be positioned in the bone, and the inner cylinder 4 inserted into the proximal end 25 of the outer cylinder 22 (i.e., the end of the device that is closest to the user). As illustrated in FIGS. 1 and 2, the inner part 2 of the bone filler device may comprise a handle 6 that can fit within a chamber 21 in the handle 24 of the outer part 20 of the bone filler device. To emplace the filler material 26 in a bone positioned at the distal end 23 of the bone filler device, the inner cylinder 4 is then pushed 37 through the outer cylinder to urge the bone filler material 26 through the outer cylinder 22 and into the bone cavity 33 in the bone 36 to be treated (FIG. 2). The inner cylinder 4 may be pushed through the outer cylinder 22 until the distal end 3 of the inner cylinder 4 is either substantially flush with the distal end 23 of the outer cylinder 22 where an end port is used for delivery of the material. Where a side port is used for delivery of the material, the inner cylinder 4 may be pushed through the outer cylinder 22 until the distal end 3 of the inner cylinder 4 is substantially flush with the distal end of the side port aperture 27. To allow for complete removal of the bone filler material 26 from the outer cylinder 22, there may be a chamber 21 in the handle 24 of the outer part shaped to allow for the inner handle 6 to sit within the chamber when the inner cylinder 4 has passed entirely through the outer cylinder 22. For example, the bone filler device may be designed such that the proximal end 9 of the inner handle 6 may be substantially flush with the proximal end 28 of outer handle 24 when the two distal ends 3, 23 of the inner and outer cylinders are aligned (see e.g., FIGS. 1 and 2).

Because the bone filler device is comprised of two straight cylindrical pieces (i.e., inner cylinder 4 and outer cylinder 22), the user of the device may require sufficient clearance to allow insertion of the inner cylinder 4 into the outer cylinder 22. It may be seen from FIG. 2 that the bone filler device may require a clearance that is equal to the sum of the distance D₁ that the outer part 20 extends from the surface 39 of the patient being treated and the length D₂ that the inner part 2 extends from the outer part 20. As described above, to ensure that the bone filler material is correctly emplaced, a radio-opaque fluorophore may be added to the filler material and the process of filling the bone monitored by transmission of X-rays using a C-arm apparatus. Optimal positioning of the C-arm (or other radio-imaging device) may require that the X-ray device be positioned as close to the patient as possible. For example, optimal positioning of the C-arm may require that the upper arm of the C-arm device be only inches from the patient's torso. Close positioning of the C arm, however, can restrict the physician's ability to insert a long inner part 2 of the bone filler device into the outer part 20 of the bone filler device.

In some cases, not all of the material 26 loaded in the outer cylinder 22 is delivered to the body part of interest. This may occur where it is not possible to determine the exact volume of material 26 that may be required. For example, it may be preferred to load an extra portion of the material into the outer cylinder, rather than having to remove a first cylinder and add additional material. Also, using a side port, the outer cylinder 22 may become immersed in, or at least partially coated by, the material 26 that is being extruded from the outer cylinder. In some cases the material being delivered comprises an adhesive component, or is highly cohesive in nature. For example, the material may comprise a cement that can harden (i.e., cure). Upon curing, the material may harden around the cannula and/or stick to the unspent material remaining in the cannula thereby making it difficult to remove the cylinder from the material that has been extruded into the body part. This may happen, for example, where the physician extrudes a majority of the material to be emplaced in the bone, and then leaves the outer cylinder in position while attending to other aspects of the procedure prior to removing the outer cylinder from the bone being treated. Thus, as illustrated in FIGS. 1 and 2, where a side port is used, a portion of the distal end of the cannula may become coated, at least in part, with the material that is being emplaced in the body part. Also, in some cases the material delivered may shrink around the cannula and/or expand into the body part leaving less room for the cannula. In this way, the cannula may become embedded in the material delivered to the body part such that it can be difficult to extricate the cannula. This can occur when there is a need to leave the cannula in place for several minutes during the procedure. The tendency of the cured material to stick to the material remaining within the cannula and/or to cure around the cannula itself may be exacerbated when the inner cylinder 4 is not pushed all the way to the end of the outer cylinder, such that there is a relatively substantial portion of the material 26 inside the cylinder (e.g., FIG. 2). Thus, it can be difficult to extricate the cannula or other delivery device from the material being emplaced in the bone or other body part. Since it can be important not to introduce trauma to the body part being repaired, there is a need to minimize the force required to remove the cannula from the body part.

Directional Access Members

Thus, in certain embodiments, the present invention comprises products and methods for delivery of a material to a body part in a subject, where the products and methods use a delivery device that reduces the tendency for the delivery device to become embedded in, or adhere to, the material being delivered to the body part. In certain embodiments, the products and methods of the present invention reduce the tendency for a device that is being used to deliver such material to become embedded in the therapeutic material once the material hardens in situ. Also, in certain embodiments, the products and methods of the present invention reduce the amount of force needed to remove the access member from the material that has been delivered to the body part. In certain embodiments, the products of the present invention may comprise a system or a kit.

For example, in certain embodiments, the present invention comprises a product comprising an access member configured to provide percutaneous surgical access to a body part in a subject. In an embodiment, the access member is configured to reduce the tendency of the access member to become embedded in the material delivered. In certain embodiments, the access member is configured to reduce the amount of force needed to remove the access member from the material that has been delivered to the body part.

The product of the present invention may comprise a side port aperture positioned near the distal end of the access member for extruding a material to a body part within a subject, wherein the access member comprises a variation in the internal diameter adjacent to the side port aperture. In an embodiment, the variation in the internal volume adjacent to the side port reduces the tendency of the material extruded from the side port to adhere to the material remaining in the access member. In this way, upon delivery of at least a portion of the material contained within the access member to the body part via the side port aperture, the material in the body part may display reduced adhesion to any material remaining within the access member upon retraction of the access member from the body part. Also, in certain embodiments, the products and methods of the present invention reduce the amount of force needed to remove the access member from the material that has been delivered to the body part.

The products of the present invention may be used with devices that are used in the art of bone repair. Thus, as described herein, the products of the present invention may be used in combination with drills and balloons for accessing the interior of certain body parts (e.g., bone) and or devices for injecting bone repair material and/or bone cement.

In an embodiment, the access member comprises a path for delivering the material to the predetermined location. The access member may comprise a distal end and a proximal end, where the distal end is the end positioned in or near the body part, and the proximal end is the end that extends outside of the subject to be accessible by the person who is carrying out the method. As described herein, the access member may provide a path to access a region or a body part that is located within a subject's body. The access member may be any type of device that can extend from the location of interest (e.g., a bone or an organ) to be accessible to a user of the access member. For example, the access member may be designed to extend from an internal body part in a subject to outside of the subject's body. The access member may comprise an elongated hollow member such as a hollow cylinder or a tube. Thus, in one embodiment, the tube may be designed to provide an access from outside of a living body to the internal body part. In an embodiment, the access member is substantially cylindrical in shape. For example, the access member may comprise a cannula, such as a cannula used to deliver a material to bone or another type of body part. One of ordinary skill in the art having the benefit of this disclosure would appreciate that the access member can be configured with other shapes and/or dimensions such as oval, hexagonal, octagonal, and the like.

In an embodiment, the access member may be configured to provide percutaneous surgical access to the predetermined location. As used herein, a percutaneous surgical access denotes passage through substantially unbroken skin, as for example, by needle puncture, a cannula or a catheter. In alternate embodiments, the percutaneous surgical access may comprise an incision ranging from about 0.05 to 8 centimeters (cm), or from about 0.1 to 4.0 cm in diameter, or from about 0.2 to 2.0 cm in diameter, or from about 0.25 to 1 cm in diameter. Or ranges within these ranges may be used. Thus, in alternate embodiments, the percutaneous surgical access may comprise an incision that is less than 4 cm in diameter, or less than 2 cm in diameter, or less than 1 cm in diameter. In one example embodiment, the percutaneous surgical access may comprise an incision of about 1 cm in diameter.

For example, in a typical percutaneous surgical repair of a spine, a cannula may establish a percutaneous path along its elongated axis to a vertebral body of one of the several vertebrae. The vertebral body extends on the anterior (i.e., front or chest) side of the vertebrae. The vertebral body comprises an exterior formed from compact cortical bone. Cortical bone is bone consisting of, relating to, or comprising the cortex or outer layer of a bony structure. The cortical bone may enclose an interior volume of reticulated cancellous or spongy, bone (also called medullary bone or trabecular bone). Cancellous bone is bone having a porous structure comprising many small cavities or cells. The vertebral body is in the shape of an oval disc, and access to the interior volume of the vertebral body can be achieved, for example, by drilling an access portal through a rear side of the vertebral body (a postero-lateral approach). The portal for the postero-lateral approach may enter at a posterior side of the vertebral body and extend anteriorly into the vertebral body. Alternatively, access into the interior volume of a vertebral body can be accomplished by drilling an access portal through one or both pedicles of the vertebra. This is known as a transpedicular approach.

The access member may have an appropriate central bore diameter and wall thickness to allow surgical instruments and or medical materials to be passed through the access member. The access member may also be strong enough to resist deformation during insertion into an interior body part, such as a bone.

The access member may be made of any material that is appropriate for use within a human or animal body. The access member may, in certain embodiments, be made of a material that is compatible with the other parts of the system. For example, the access member may be made of metal such as aluminum, stainless surgical steel, spring steel, a nickel titanium alloy or other alloys. Or, the access member may be made of plastic, such as polypropylene, polyurethane, polyethylene, Torque (stacked) coil made out of a metal such as stainless steel, polyethyleneteraphthalate (PET), TEFLON®, ionomer, polycarbonate or nylon. Or, the access member may be made of silicates or liquid crystal polymers. One of ordinary skill in the art having the benefit of this disclosure would appreciate that other materials, including those that are well-known to one in the art, may be applied to configure the access member described herein. In certain embodiments, the access member may be made of two types of materials. For example, in one embodiment, a portion of the distal end of the access member that includes the side port aperture is made of a plastic (e.g., TEFLON®, PET, ionomer, polyurethane, polycarbonate or nylon), while the more proximal end of the access member is made of metal.

In an embodiment, the access member may be coated with a substance that has the ability to shrink and expand. The coating substance may then provide a cushion, such that if a material being delivered to a body part hardens around the access member, the cushioning substance will absorb most of the shrinkage induced by the cured material. In an embodiment, the coating substance has a low frictional coefficient (i.e., is slippery). In an embodiment, the coating substance is made of TEFLON® (e.g., PFA from Dupont), silicon, polyethylene, polyurethane, and the like. In certain embodiments, the access member may be coated with any appropriate medical grade coating including an anti-infective, an anti-coagulant, a release coating, and/or a slipping agent (e.g., silicone or other slipping agents, or commercially available contains such as MEDCOAT 2000™, LubriLAST™, EPOSTAR and the like).

The product may further comprise a device to be inserted into the access member for urging the material through the access member to the body part of interest. In an embodiment, the device for urging the material through the access member comprises a inner member, such as the cylinder 4 shown in FIGS. 1 and 2. For example, a rod, plunger, or stylet having a diameter slightly smaller than the internal diameter of the access member may be used. The inner member may be substantially rigid, or may be flexible as discussed in more detail below. Materials used for the inner cylinder may be any material that is appropriate for use within a human or animal body. The inner member may be made of the same material as the access member. Such materials may include metal such as aluminum, stainless surgical steel, spring steel, a nickel titanium alloy or other alloys. Or, the inner member may be made of plastic, such as polypropylene, polyurethane, polyethylene, polyethyleneteraphthalate (PET), TEFLON®, ionomer, polycarbonate or nylon. Or, the inner member may be made of silicates or liquid crystal polymers. In certain embodiments, the inner member may be coated with any appropriate medical grade coating including an anti-infective, an anti-coagulant, a release coating, and/or a slipping agent.

The access member may comprise a variety of means to reduce the adherence of the material that has already been extruded to the material that remains within the access member, and/or the tendency of the access member to become embedded in the material that has been delivered to the body part.

In an embodiment, the access member comprises a variation in the internal diameter of the access member adjacent to at least a portion of the side port. In certain embodiments, the variation may comprise a reduction in the inner volume or diameter for a portion of the access member. In one embodiment, the variation in the internal diameter adjacent to the side port aperture comprises a reduction in the internal diameter at the distal end of the side port aperture as compared to the proximal end of the side port aperture. For example, the variation may reduce the diameter of the access member at the distal end of the side port such that the cross-section of the material as it exits the side port is smaller at the distal end than at the proximal end.

The variation in the internal diameter adjacent to the side port aperture may comprise a variety of shapes. In certain embodiments, the variation comprises an incline from the inner wall that is about opposite the proximal end of the side port aperture to the inner wall that abuts the distal end of the side port aperture. For example, in certain embodiments, the variation in the internal diameter adjacent to the side port aperture comprises at least one of a straight ramp, a convex ramp, a compound convex ramp, a concave ramp, a compound concave ramp, a step, an angled step, a reverse angled step, a plurality of surfaces comprising at least one convex surface and one concave surface. Or similar shapes and/or combinations of such shapes may be used. For example, in certain embodiments, the variation in the internal diameter adjacent to the side port aperture may comprise a plurality of steps, a wave or plurality of waves, or combinations thereof. Or, similar shapes may be used.

In an embodiment, the portion of the access member that provides a variation in the internal volume adjacent to the side port aperture may be coated with a substance that has the ability to shrink and expand. The coating substance may then provide a cushion, such that if a material being delivered to a body part hardens, the cushioning substance will absorb most of the shrinkage induced by the cured material. In an embodiment, the coating substance has a low frictional coefficient (i.e., is slippery). In an embodiment, the coating substance is made of TEFLON® (e.g., PFA from Dupont), silicon, polyethylene, polyurethane, and the like. In certain embodiments, the portion of the access member that provides a variation in the internal volume adjacent to the side port aperture may be coated with any appropriate medical grade coating including an anti-infective, an anti-coagulant, a release coating, and/or a slipping agent.

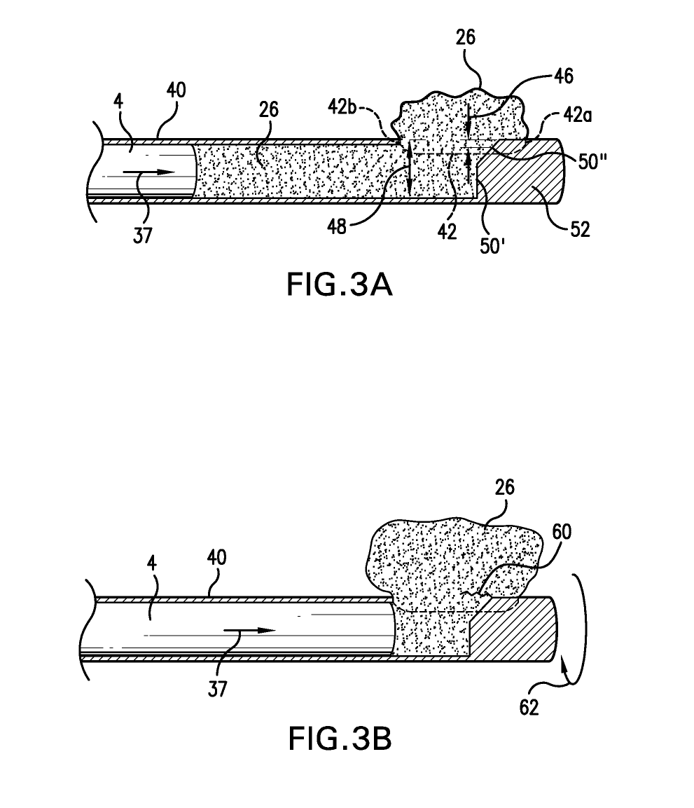

The present invention may be better understood by reference to the example embodiments illustrated in the Figures. For example, in an embodiment, the access member may comprise an angled step (i.e., having the horizontal surface of the step be angled) at the point where the material exits the access member as shown in FIG. 3, panels A and B. Thus, in an embodiment, the present invention comprises an access member 40 (e.g., a cannula) having a side port aperture 42, and an angled step 52 positioned such that the inner diameter (ID) 46 of the access member is less at one end of the side port 42a than the inner diameter 48 at the other end of the side port 42b. As shown in FIG. 3, the angled step comprises a first vertical surface 50′ with a more distal sloped surface 50″, to make a step with an angled surface. In this way, there may be a smaller cross-section or volume of the material being extruded 26 at the distal end of the side port aperture of the access member. By having a reduced cross-section of the material at the distal end of the opening, there may be a less adherence of the material being extruded to the material remaining within the access member at that point since there is a reduced amount of material at that part of the side port aperture to bind to the extruded material. In an embodiment, repositioning of the access member, as for example by gentle twisting, pulling, jiggling, or the like, of the access member 62, may induce a break in the material 60 (FIG. 3B).

The discontinuity (e.g., variation of the internal diameter) adjacent to the side port may comprise a built-in feature of the access member. Alternatively or additionally, the discontinuity within the access member may comprise an accessory feature, such as a plug or other fixture that can be inserted into a substantially hollow end of an access member.

Thus, in other embodiments, the present invention comprises a product comprising a removable structure (e.g., an insert) for use with an access member having a side port for extruding a material to a predetermined location within a subject. In certain embodiments, the present invention comprises a product comprising an insert to be emplaced in the internal volume of an access member. In certain embodiments, the access member may be configured to provide percutaneous surgical access to a body part in a subject, and with a distal end for insertion into a body part and a proximal end for access by a user, and a side port aperture positioned at or near the distal end of the access member for extruding a material to a body part within the subject.

In certain embodiment, the structure is designed to reduce the adhesion of the material remaining in the access member to material delivered to the body part and/or the tendency of the access member to become embedded in the material delivered to the body part. In an embodiment, the structure is designed such that insertion of the structure into the access member provides a variation in the internal diameter adjacent to the side port aperture. In an embodiment, the variation in the internal volume adjacent to the side port provided by the structure reduces the tendency of the material extruded from the side port to adhere to the material remaining in the access member. In this way, upon delivery of at least a portion of the material contained within the access member to the body part via the side port aperture, the material in the body part may display reduced adhesion to any material remaining within the access member upon retraction of the access member from the body part. Also, in certain embodiments, the inserting the structure into the access member reduces the amount of force required to remove the access member from the material emplaced in the body part.

Once inserted, the structure may be fixedly attached to the access member, or may be removably attached to the access member. The structure may thus fit within the access member to reduce adhesion of the material being delivered by access member to the material that remains in the access member, or to the access member itself.

The insert may comprise a variety of means to reduce the adherence of the material that has already been extruded to the material that remains within the access member, or the tendency of the access member to become embedded in the material. In an embodiment, the insert provides a variation in the internal diameter of the access member adjacent to at least a portion of the side port. In certain embodiments, the variation may comprise a reduction in the inner volume or diameter for a portion of the access member. In one embodiment, the variation in the internal diameter adjacent to the side port aperture comprises a reduction in the internal diameter at the distal end of the side port aperture as compared to the proximal end of the side port aperture.

The structure for positioning adjacent to the side port aperture may comprise a variety of shapes. For example, in certain embodiments, the structure may comprise at least one of a straight ramp, a convex ramp, a compound convex ramp, a concave ramp, a compound concave ramp, a step, an angled step, a plurality of surfaces comprising at least one convex surface and one concave surface. In certain embodiments, the variation comprises an incline from the inner wall that is about opposite the proximal end of the side port aperture to the inner wall that abuts the distal end of the side port aperture. Or similar shapes and/or combinations of such shapes may be used. The structure may also comprise, in alternate embodiments a plurality of steps, a wave or plurality of waves, or combinations thereof. Or, similar shapes may be used.

For example, in an embodiment, the insertable structure may comprises an angled step, such that positioning the angled step adjacent to the side port reduces the internal volume of the access member at the distal end of the side port. Alternatively, the insertable structure may comprise at least one vertical step, such that positioning the at least one step adjacent to the side port reduces the internal volume of the access member at the distal end of the side port.

In one embodiment, insertable structure may be coated at least in part, with a substance that has the ability to shrink and expand. In certain embodiment, the portion of the structure that abuts the side port opening (i.e., such as the surface of the ramp, steps, or waves that faces the opening) is coated. The coating substance may then provide a cushion, such that if a material being delivered to a body part hardens around the access member, the cushioning substance will absorb most of the shrinkage induced by the cured material. In an embodiment, the coating substance has a low frictional coefficient (i.e., is slippery). In an embodiment, the coating substance is made of TEFLON® (e.g., PFA from Dupont), silicon, polyethylene, polyurethane, and the like. In certain embodiments, the access member may be coated with any appropriate medical grade coating including an anti-infective, an anti-coagulant, a release coating, and/or a slipping agent.

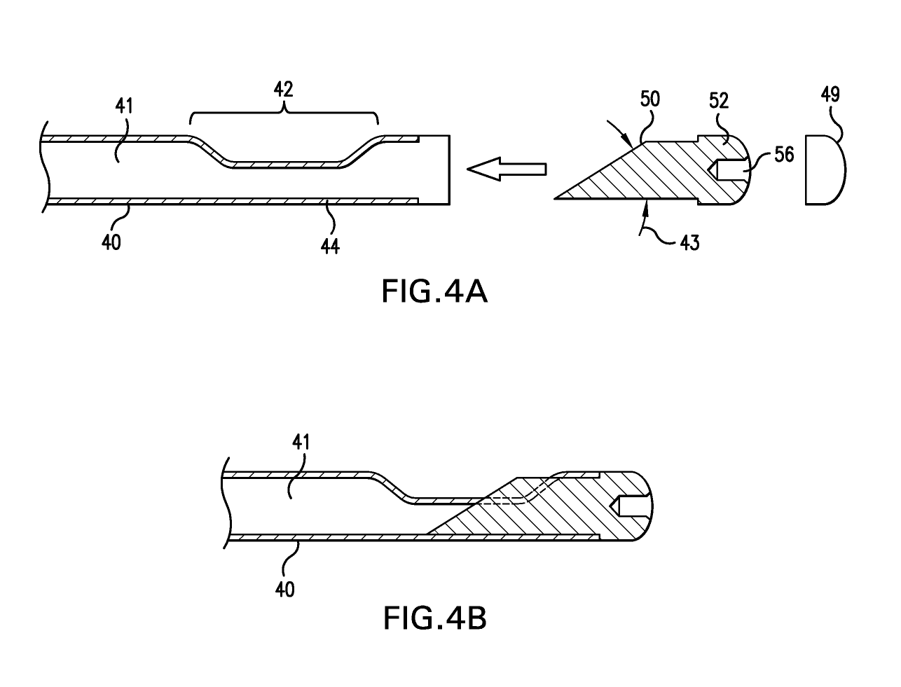

For example, as illustrated in FIG. 4, in an embodiment, the distal end of an access member may comprise a cap 49 that may be removed (e.g., unscrewed) to access the inner volume 41 of the access member 40 (FIG. 4A). Once the cap has been removed, a plug 52 comprising a ramp 50 may be inserted into the access member 40 (FIG. 4B). In one embodiment, the distal end of the plug replaces the cap to form the distal end of the access member.

The structure for introducing a variation in the internal diameter of the access member may comprise a variety of shapes. In an embodiment, the structure for introducing a discontinuity in the access member may comprise a ramp. The ramp may be positioned adjacent to the side port so as to reduce the inner volume of the access member at the distal end of the side port compared to the proximal end of the side port. The surface of the ramp may comprise a variety of slopes depending upon the nature of the access member. Thus, the ramp may comprise a slope 43 (FIG. 4) having an angle of from about 2 degree to 90 degrees, or from about 5 degrees to about 80 degrees, or from about 10 degrees to about 70 degrees, or from about 10 degrees to about 60 degrees, or from about 15 degrees to about 50 degrees, or from about 20 degrees to about 35 degrees, or from about 20 degrees to about 30 degrees. Or, ranges within these ranges may be used.

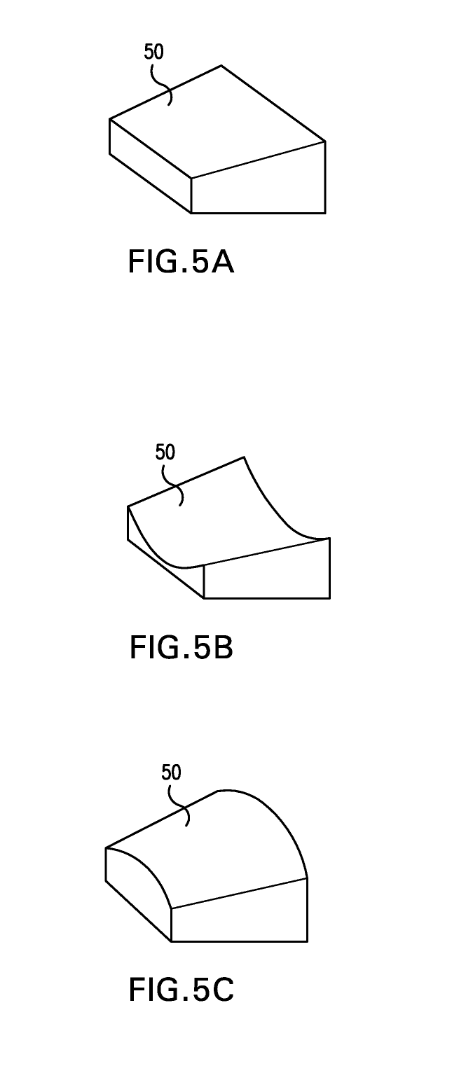

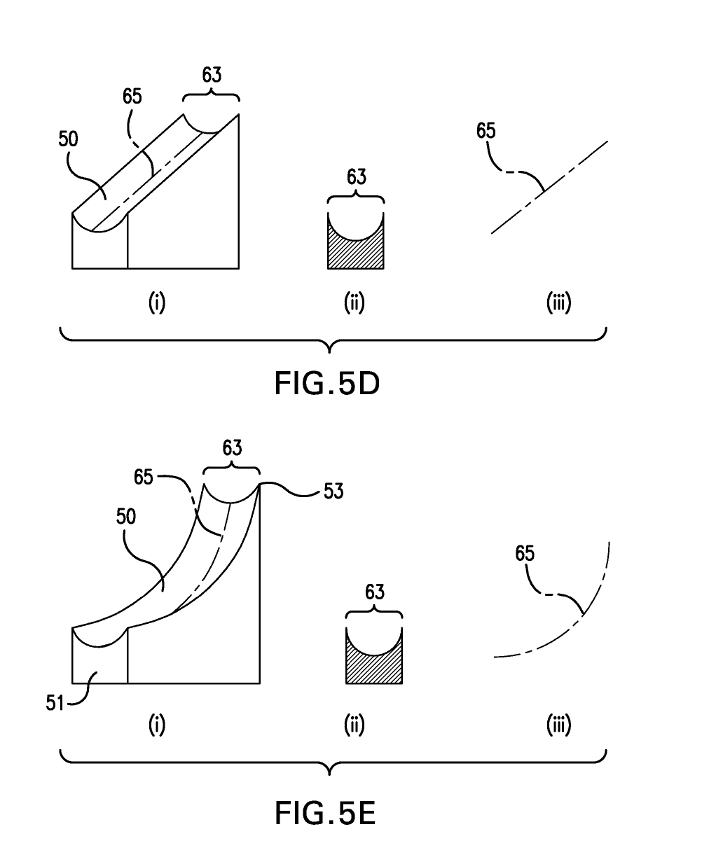

In another embodiment (e.g., FIG. 5) the structure positioned adjacent to the opening may comprise an angled step. In an embodiment, the surface of the ramp or angled step 50 may be substantially flat (FIG. 5A). Alternatively, the surface of the ramp or angled step may comprise a concave shape (FIG. 5B). In other embodiments, the surface of the ramp or angled step may comprise a convex shape (FIG. 5C).

In one embodiment, the ramp or angled step 50 may comprise a compound angle. FIGS. 5D and 5E show an example of a standard concave surface and a compound concave surface, respectively. For a standard concave surface, the surface 50 although concave across the width of the surface 63; see e.g., (ii) of FIGS. 5D and 5E) has a constant slope along the length of the surface 65 (i.e., see (iii) of FIG. 5D). For a compound surface, the surface has a changing slope along both the width of the piece and the length of the piece. Thus, it can be seen that a compound concave surface has surface 50 that varies in slope 65 along the length of the angled step or ramp so as to vary in slope from the distal end 53 to the proximal end 51 of the angled step (FIG. 5E). Variations of such compound angles (e.g. a concave or convex wave) may be used as either insertable plugs 52 or built-in to the access members of the present invention.

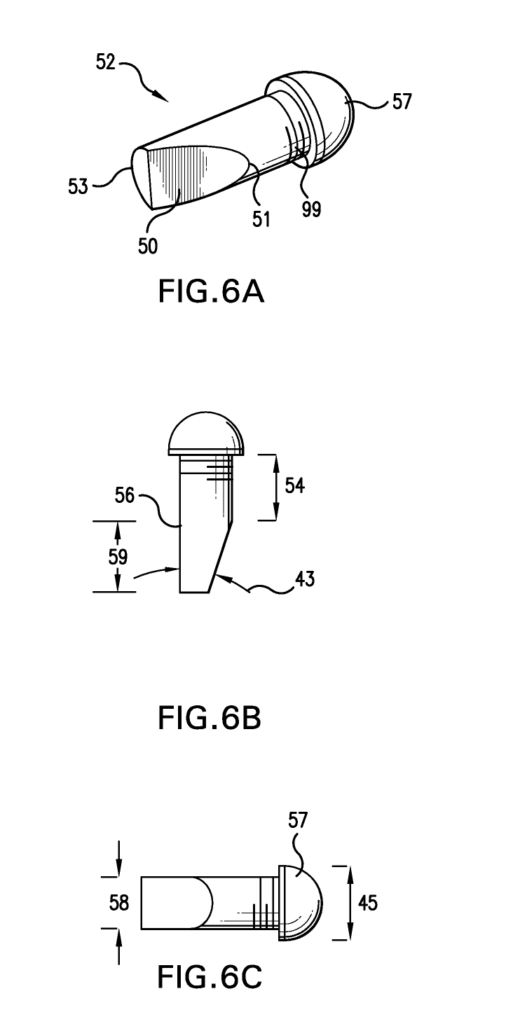

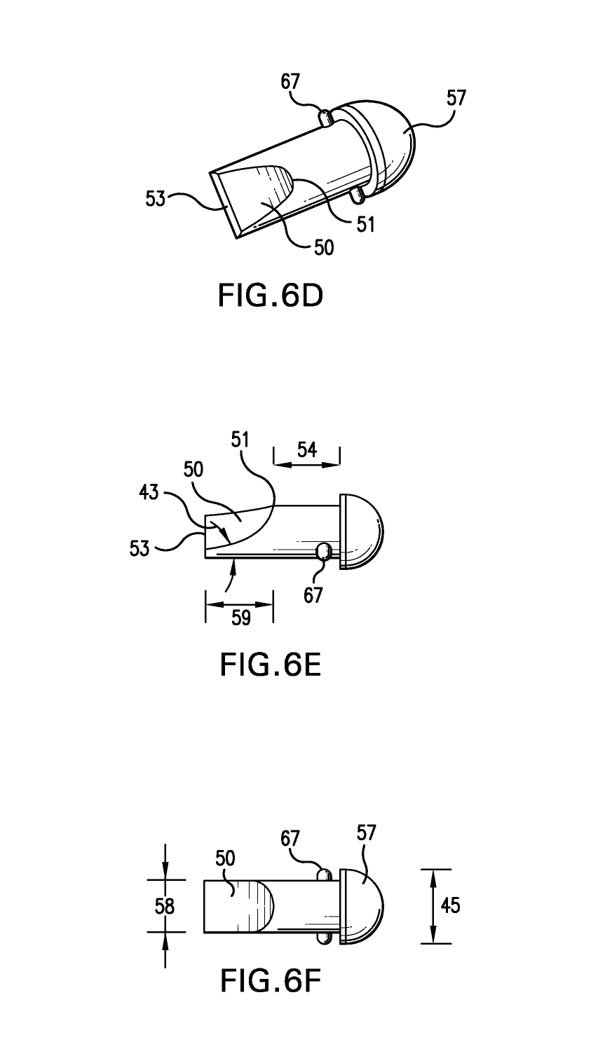

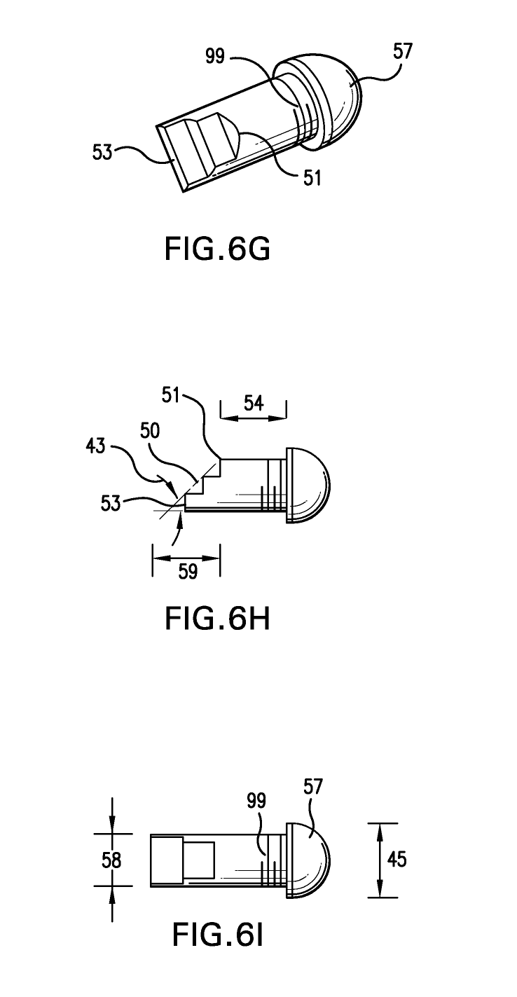

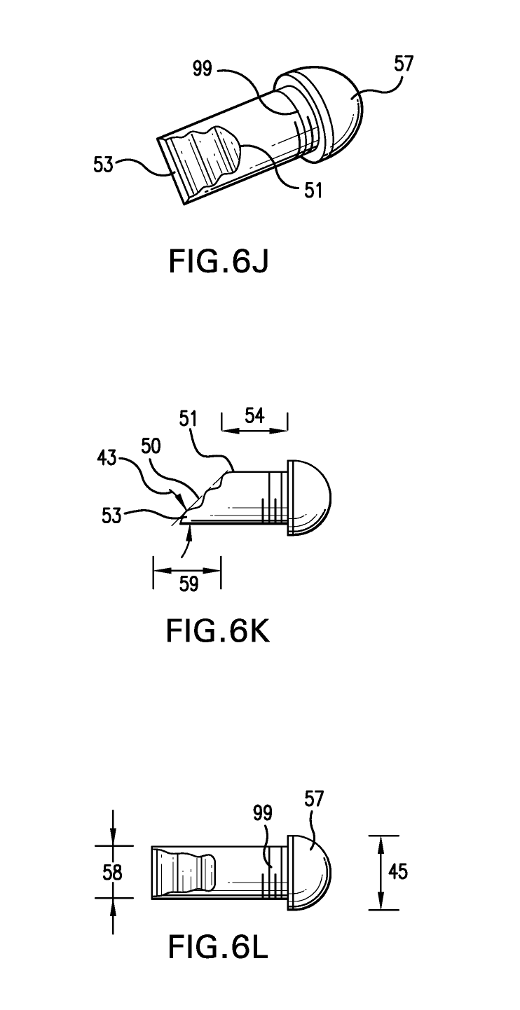

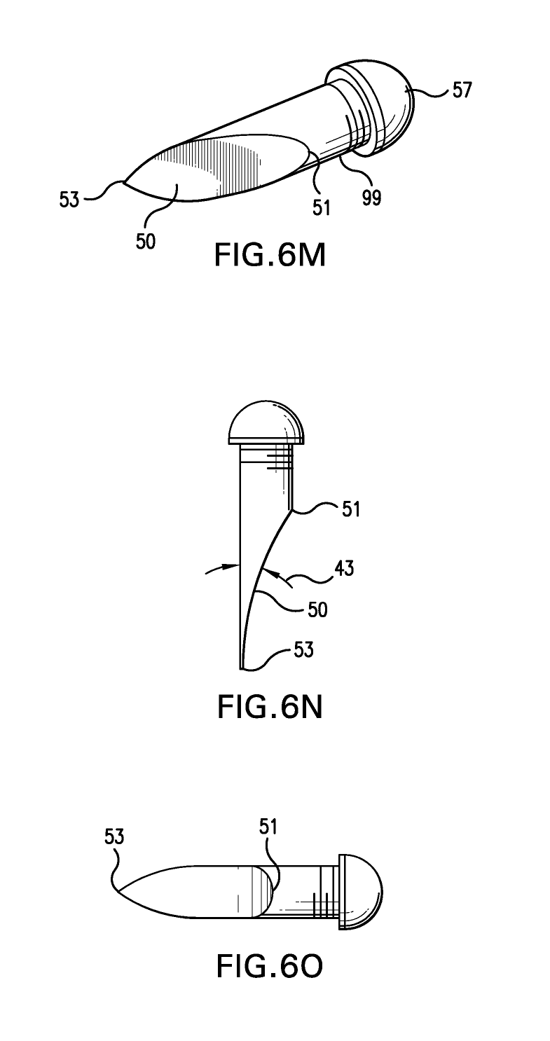

For example, in alternative embodiments, an insertable angled step 52 having a substantially flat surface 50, and distal 51 and proximal ends 53, may be machined as a separate part to be inserted in the access member (FIG. 6A-C). The angled step 52 may comprise a cylindrical distal portion 54 that fits within the inner volume of the access member. In an embodiment, the angled step 52 may also comprise a distal end 57 that can be substituted for the distal end (e.g., cap 49) of the access member 40. In alternate embodiments, the angled step may comprise a surface 50 that ranges in slope from about 2 degree to 90 degrees, or from about 5 degrees to about 80 degrees, or from about 10 degrees to about 70 degrees, or from about 10 degrees to about 60 degrees, or from about 15 degrees to about 50 degrees, or from about 20 degrees to about 35 degrees, or from about 20 degrees to about 30 degrees (FIG. 6B). Or, ranges within these ranges may be used.

In another embodiment, an insertable ramp or angled step having a substantially concave surface 50 may be machined as a separate part to be inserted in the access member (FIG. 6D). Similar to a flat (i.e., straight) ramp or angled step, the concave ramp or angled step may comprise cylindrical distal portion 54 that fits within the inner volume of the access member. The concave surface may, in certain embodiments, comprise a compound concave surface. In an embodiment, the insertable ramp or angled step 52 may also comprise a distal end 57 that can be substituted for the distal end (e.g., cap 49) of the access member 40. In alternate embodiments, the ramp may comprise a surface 50 that ranges in the overall slope from about 2 degree to 90 degrees, or from about 5 degrees to about 80 degrees, or from about 10 degrees to about 70 degrees, or from about 10 degrees to about 60 degrees, or from about 15 degrees to about 50 degrees, or from about 20 degrees to about 35 degrees, or from about 20 degrees to about 30 degrees (FIG. 6D). Or, ranges within these ranges may be used. For example, in some embodiments of a compound concave surface, the slope of the concave ramp or angled step may average to about 37.5 degrees, but is shallower at the distal end 53 and more steep at the proximal end 51 (i.e., a compound concave curve).

A variety of shapes may be used for the insertable plugs of the present invention. For example, in an embodiment, the plug may comprise a plurality of steps (see e.g., FIGS. 6G-6T) or at least one convex and one concave surface so as to form a wave or a series of waves (see e.g., FIG. 6J-L) that together comprise a defined overall slope 50. In an another embodiment, the insertable plug may comprise a convex surface 50, but may have a tapered base 53 as shown in FIGS. 6M, 6N and 60.

In an embodiment, the piece 52 is machined to fit snugly within the inner volume of an access member used to deliver material to a bone. For example, in alternate embodiments, the length of the base (i.e., the total internal length) of the insertable plug 56 may range from about 0.1 to 2.0 inches, or from about 0.1 to 1.0 inch, 0.1 to 0.6 inches, or from about 0.2 to about 0.5 inches, or from about 0.2 to about 0.45 inches, or may be about 0.3 to 0.4 inches. To fit within the distal end of a bone filler device, the distal portion of the insert 54 may in alternate embodiments, range in length 54 from about 0.02 to 1.0 inch, or from about 0.02 to 0.5 inches, or from about 0.03 to 0.4 inches, or from about 0.03 to 0.2 inches, or from about 0.04 to 0.15 inches, or from about 0.06 to about 0.12 inches, or from about 0.07 to about 0.1 inches, or may be about 0.08 to 009 inches. Or, ranges within these ranges may be used.

Also, in alternate embodiments, the width of the insertable ramp is such that when inserted in the access member 40, there is no detectable space between the inner wall of the access member and the outer wall of the insertable ramp 52. Thus, for a bone filler device, the outside diameter (width) 58 of the insertable plug may range from about 0.4 to about 0.02 inches, or from about 0.2 to about 0.04 inches, or from about 0.135 to about 0.085 inches, to about 0.125 to about 0.095 inches, to about 0.115 to about 0.105 inches, or may be about 0.111 inches. Or ranges within these ranges may be used. Where the insertable piece includes a distal end 47 that may replace the distal end of the access member, the end of the insertable piece may be sized to be the same circumference as the end of the access member. Thus, in alternate embodiments, the outside diameter (width) 45 of the distal end of the insertable plug (or piece) may range from about 0.6 to about 0.03 inches, or from about 0.4 to about 0.05 inches, or from about 0.165 inches to about 0.109 inches, or from about 0.140 inches to about 0.120 inches, or from about 0.135 inches to about 0.130 inches, or may be about 0.134 inches. Or, ranges within these ranges may be used.

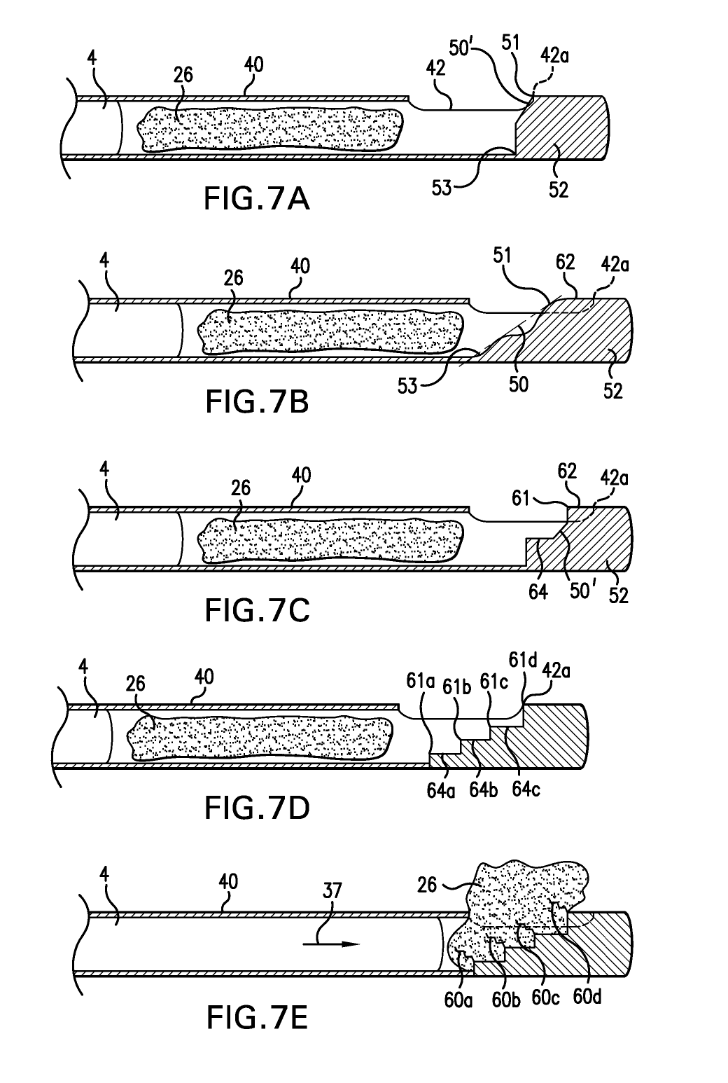

In an embodiment the distal end 51 of the fixture used to introduce a variation in the internal diameter of the access member may substantially coincide with the distal end 42a of the side port aperture 42 (FIG. 7A). In alternate embodiments, the distal end 51 may be positioned proximal to the distal end of the opening so as to create portion 62 that is essentially co-linear with the external surface access member (FIG. 7B). In certain embodiments, the fixture may create a point of discontinuity 61 at which material exiting the access member may initiate a fracture upon the user repositioning the access member as described in more detail herein. For example, in alternate embodiments, the user may reposition the access member by at least one of twisting, jiggling, pulling on (i.e., pulling the access member in a proximal direction) or otherwise moving the distal end of the access member in relation to the material that is delivered to the body part. For example, in an embodiment, an angled step that has its incline positioned at the distal end of the opening may be used (FIG. 7A). Or, the fixture may comprise a plurality of waves comprising a series of discontinuities at which material exiting the access member may initiate a fracture upon the user twisting the access member as described in more detail herein (FIG. 7B).

Alternatively, the discontinuity in the access member may comprise a square step or a reverse angled step 64 (FIG. 7C). Or, a plurality of steps 64a, 64b, 64c may be used (FIG. 7D). In this way, as the material 26 is urged out of the opening 42 by the movement 37 of the inner cylinder 4, the device may comprise a point 61 (FIG. 7C) or a series of discontinuous points 61a, 61b, 61c, and 61d (FIG. 7D) which the material 26 being extruded may encounter. These discontinuous regions of the access member may then induce a region of discontinuity in the material being extruded, resulting in reduced adherance of the extruded material to the access member or the material therein, and/or a point or points (e.g., 60a, 60b, 60c and 60d in FIG. 7E) at which the material may fracture so as to be separated from the material remaining in the access member.

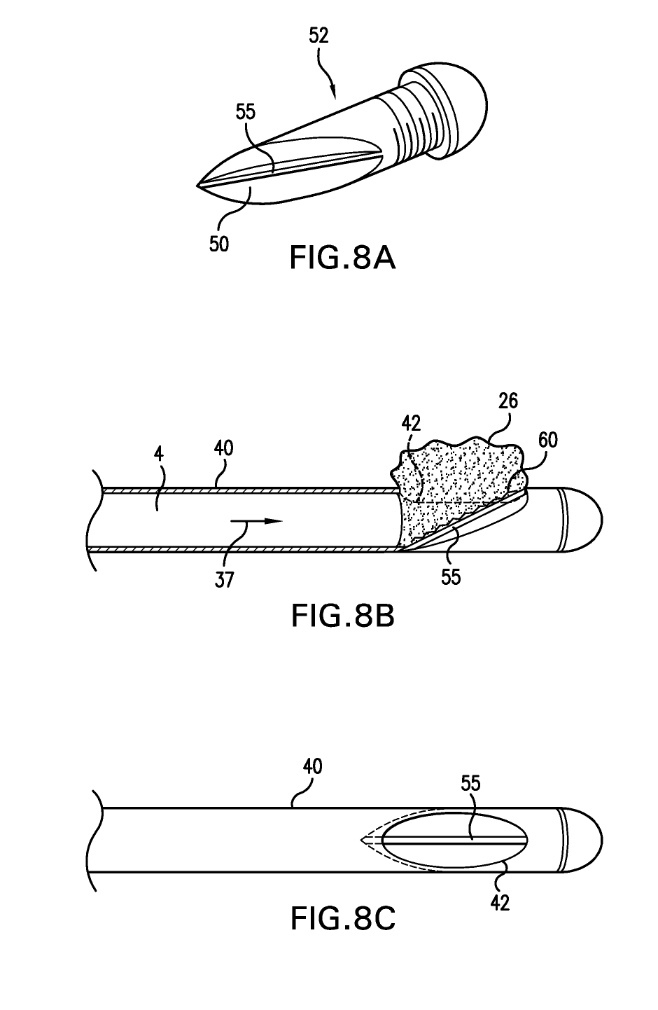

In another embodiment, an insertable ramp 52 comprising a dividing element 55 (e.g., a fin) may be used to provide a ramp that has a portion for inducing a region of discontinuity along the length of the dividing element (FIG. 8A-8C). Thus, as the material is extruded from the access member, the portion of the material adjacent to the dividing element 55 may comprise a region of reduced adherance 60 to the insert 52. Thus, the material have a region of discontinuity or may tend to fracture at this region as the access member is pulled away from the emplaced material (FIG. 8B).

In the embodiment where the structure is an accessory feature, the insertable structure may comprise an element to position the fixture adjacent to the side port. For example, the insertable structure may comprise an element to position the structure adjacent to the side port so that the structure has the correct alignment with respect to the length of the access member as well as being in the correct orientation to the side port opening. For example, in the case of the an angled step, the plug may be positioned such that step extends from the distal end of the side port opening to the inner wall that is opposite to the proximal end of the opening as shown in FIGS. 7A and 7D. In an embodiment, the structure may comprise threads 99, such that the structure is threaded into an access member after removing the removable cap, similar to a screw being threaded into a housing (see e.g., FIG. 6A-6C). Or, the structure may comprise pins 67 (see FIG. 6D-6F), or another type of element that may be used to align the structure (e.g., the ramp or steps) with the side port of the access member. For example, the pins may be fashioned so they can be pushed in (e.g., having a spring mechanism) to allow for insertion of the ramp or other structure into the access member, but which can extend outwardly (or spring) into a hole or other type of receptacle that has been fashioned in the inner wall of the access member. Or other types of alignment mechanisms may be used to ensure that the structure is properly aligned along the length of the access member. For example, in an embodiment, the end piece 57 of the insertable ramp (e.g., FIGS. 4A and 4B) is fashioned so as to limit the distance that the insertable structure may extend into the inner volume of the access member and thus, may be used to align the ramp along the longitudinal axis of the access member. For example, in the case of an angled step, the step may be positioned such that step extends from a point that is positioned at about to the distal end of the opening as shown in FIG. 7A. Or, the fixture may comprise a plurality of discontinuities such as the wave, reverse angled step, and multiples steps shown in FIGS. 7B, 7C, and 7D.

The structure or portion of the access member that induces reduced adherence of the extruded material to the material remaining in the access member may be made of any material that is appropriate for use within a human or animal body. In certain embodiments, a structure used to introduce a variation in the inner volume of the access member may, in certain embodiments, be made of a material that is compatible with the other parts of the system. For example, the structure may be made of metal such as aluminum, stainless steel, stainless surgical steel, spring steel, a nickel titanium alloy or other alloys. Or, the structure may be made of plastic, such as polypropylene, polyethylene, polyethyleneteraphthalate (PET), TEFLON®, ionomer, polycarbonate or nylon. Or, the structure may be made of silicates or liquid crystal polymers. In certain embodiments, the structure may be coated with any appropriate medical grade coating including an anti-infective, an anti-coagulant, a release coating, and/or a slipping agent. One of ordinary skill in the art having the benefit of this disclosure would appreciate that other materials, including those that are well-known to one in the art, may be applied to configure the structure used to decrease the tendency of the access member to become embedded in the material being delivered to a body part described herein.

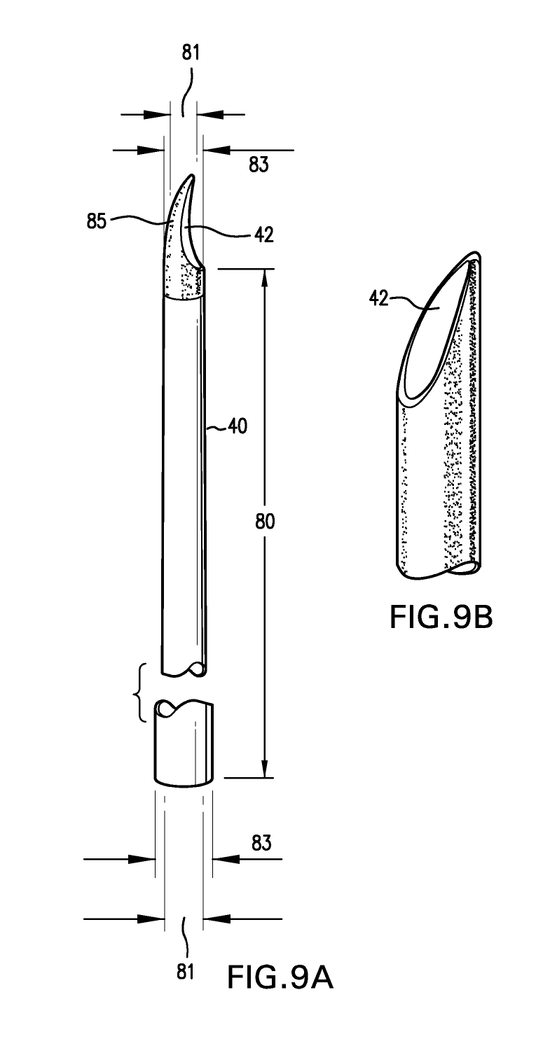

In yet another embodiment, the shape of the access member provides a means to decrease adherence of the material being delivered to a body part to the material remaining in the access member or to reduce the tendency of the access member to become embedded in the material delivered to the body part. Also, in certain embodiments, the shape of the access member reduces the amount of force needed to remove the access member from the material that has been delivered to the body part. Thus, in certain embodiments, the access member may comprise a reduction in the external cross-section of at least a portion of the distal end of the access member that includes the side port aperture. In an embodiment, the access member comprises a radius that exhibits a reduction in size along the length 80 of the access member, such that the access member has a smaller radius at the distal end (i.e., the end positioned closest to the body part and comprising opening 42) than at the proximal end (e.g., FIG. 9A). In an embodiment, both the internal radius 81, and the external radius 83 decrease from the proximal end to the distal end of the access member. Also, in an embodiment, the access member may comprise an internal radius 81, and external radius 83 which is significantly narrowed adjacent to the opening 42. In other embodiments, the access member has a constant diameter throughout most of the length of the access member, but is shaped at the distal tip to comprise a reduced cross-section beginning at about the point where the side port aperture begins and continuing such that the distal end of the access member has a cross-section that is substantially reduced as compared to the proximal end of the access member. For example, in certain embodiments, the outer diameter at the proximal end of the access member is about 0.134 inches and the OD at the distal end is less than 0.12, 0.1, 0.08, or 0.06, or 0.04 inches. Or, other ranges may be used depending upon the nature of the access member and the body part being accessed.

Additionally or alternatively, the external surface of the access member may be curved so as to aid in removal of the access member from the material deposited in the body part. For example, the access member may comprise a concave outer surface 85 that forms the back side of the side port aperture. In certain embodiments, the concave outer surface 85 may mimic the concave curvature of the inner surface of the access member that is adjacent to the side port aperture 42 (FIG. 9A). In an embodiment, the curved and tapered outer surface extends to a narrow tip at the end of the access member thereby reducing the tendency of the access member to become embedded in the material that has been extruded into the body part. Also, the curved tip may facilitate extricating the access member from the material as there may be an area adjacent to the curved surface 85 that is not embedded in the material, but that comprises a small void in the material.

Or, the access member may comprise a tapered distal end, but have a substantially flat surface that forms the back side of the side port aperture (FIG. 9B). In an embodiment, the tapered outer surface extends to a narrow tip at the end of the access member thereby reducing the tendency of the access member to become embedded in the material that has been extruded into the body part. Also, the tapered tip may facilitate extricating the access member from the material as there may be an area adjacent to the tapered surface that is not embedded in the material, but that comprises a small void in the material.

In another embodiment, the access member may comprise an indentation along the outer surface which provides a convex curvature to the internal radius of the access member. In this embodiment, a material may be directed out of the access member and away from the back side of the opening. Thus, there may be a reduced tendency for the material to cure around the back side of the access member. Additionally, the tapered shape of the access member may facilitate creating a discontinuity or void in the material being delivered to the body part.

In certain embodiments, the access member may be coated with a material to help reduce adhesion of the access member to the material being extruded from the access member. In an embodiment, at least a portion of the distal end of the access member that includes the side port aperture is coated with a plastic. In one embodiment, access member may be coated with a substance that has the ability to shrink and expand. The coating substance may then provide a cushion, such that if a material being delivered to a body part hardens around the access member, the cushioning substance will absorb most of the shrinkage induced by the cured material. In an embodiment, the coating substance has a low frictional coefficient (i.e., is slippery). In an embodiment, the coating substance is made of TEFLON® (e.g., PFA from Dupont), silicon, polyethylene, polyurethane, and the like. In certain embodiments, the access member may be coated with any appropriate medical grade coating including an anti-infective, an anti-coagulant, a release coating, and/or a slipping agent.

In other embodiments, the side port aperture itself may be used to provide a means by which to induce reduced adherence of the material being delivered to a body part to any material remaining within the access member and/or to reduce the tendency of the access member to become embedded in the material delivered to the body part. Also, in certain embodiments, the side port aperture may be designed to reduce the amount of force needed to remove the access member from the material that has been delivered to the body part. In certain embodiments, the side port aperture may comprise a shape such that upon delivery of at least a portion of the material contained within the access member to the body part via the side port aperture, the shape of the side port aperture reduces the tendency of the material extruded from the side port to adhere to the material remaining in the access member. Thus, in certain embodiments, the side port aperture is shaped such that the adhesive material in the body part displays less adhesion to the access member and/or any material remaining within the access member upon retraction of the access member from the body part than when a round or oval side port aperture is used. For example, the side port may be shaped such that a discontinuity is introduced into the material at certain points as the material exits the access member. In one embodiment, the side port may be shaped such that the material is sheared at least in part at certain points as it exits the access member. Or, the side port may be shaped such that a fracture point is induced in the material at certain points as the material exits the access member.

For example, the side port may be shaped such that there is a point at which the material is forced through an aperture that comprises series of points or edges. In this way, the material may easily fracture as the access member is twisted away from the extruded or emplaced material. Thus, in certain embodiments, the access member may comprise a discontinuity in the shape of the side port. In certain embodiments, the side port comprises at least one of a serrated edge. Or, the side port aperture may comprise a discontinuity in the shape of aperture such that the width of at least one portion of the aperture is substantially reduced in size as compared to the width at another portion of the aperture. Or, the side port aperture may comprise a division of the aperture into a plurality of apertures. Alternatively or additionally, there may be a portion of the side port that comprises a plurality of serrated or straight edges.

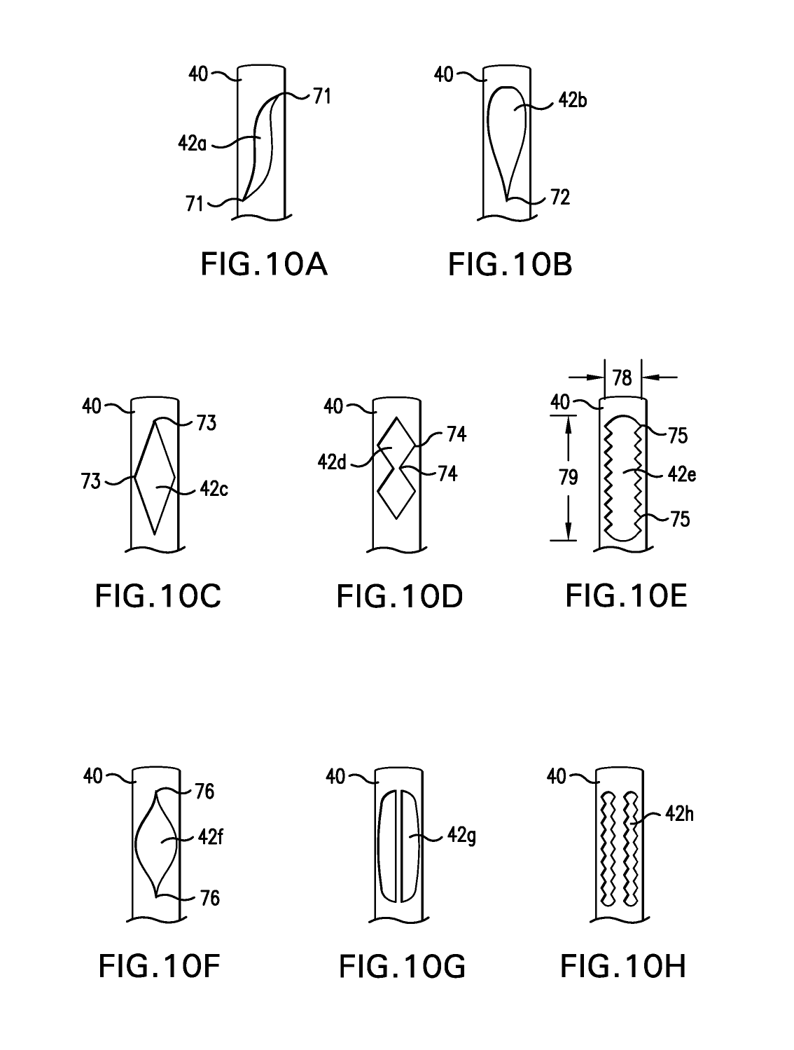

FIG. 10 shows examples of side ports that may be used to introduce a discontinuity in a material as the material is urged through the side port so as to introduce reduced cohesiveness, such that the material may sever, shear or break at such points of discontinuity. For example, the access member may comprise a side port 42a shaped as a compound curve (FIG. 10A). In an embodiment of a compound curve shaped aperture, a discontinuity may be introduced in the material such that the material may shear or fracture at each of the end points of the aperture 71 as the access member is repositioned (e.g., twisted away from the emplaced material) Or, the access member may comprise a side port 42b in the shape of a tear drop (FIG. 10B). In an embodiment of a tear drop shaped aperture, a discontinuity may be introduced in the material at the end points of the aperture 72 as the access member is repositioned away from the emplaced material. In another embodiment, the access member may comprise a side port 42c that is shaped like a diamond (FIG. 10C). In an embodiment of a diamond-shaped side port aperture, a discontinuity may be introduced in the material at the various points 73 of the diamond as the access member is repositioned away from the emplaced material. Alternatively, the access member may comprise a side port 42d that is shaped like two (or more) interlaced diamonds (FIG. 10D), such that a discontinuity may be introduced in the material at the various points 74 of the diamonds as the access member is repositioned away from the emplaced material. Alternatively, the access member may comprise a side port 42e that is substantially oval with serrated edges (FIG. 10E). In an embodiment of a serrated side port, a discontinuity may be introduced in the material such that the material may shear or fracture at the various points 75 of the serrated edges as the access member is repositioned away from the emplaced material. Or, the access member may comprise a side port 42f that is shaped like an eye (FIG. 10F) such that a discontinuity is introduced in the material may at the end points 76 of the eye as the access member is twisted away from the emplaced material. Or, a bifurcated side port 42g, or serrated bifurcated side port 42h, may be used (FIGS. 10G and 10H, respectively).

The side port may comprise a length and width similar to that used for a standard side port. Thus, in alternate embodiments, the width 78 of the side port 42 may range from about 0.4 to about 0.04 inches, or 0.2 to about 0.05 inches, or from about 0.135 to about 0.085 inches, to about 0.125 to about 0.095 inches, to about 0.115 to about 0.105 inches, or may be about 0.111 inches. Also, the length 79 of the side port 42 may range from about 0.08 to about 2 inches, or from about 0.1 to about 1 inch, or from about 0.15 to 0.6 inches, or from about 0.2 to about 0.5 inches, or from about 0.25 to about 0.4 inches, or is about 0.35 or 0.36 inches (FIG. 10E). Or, ranges within these ranges may be used. In certain embodiment, the side port comprises a shape such that the length of the opening is greater than the diameter of the tube, so as to allow the material to exit the tube without any build up of pressure at the distal end of the tube.

In an embodiment, the products of the present invention may be used with a cement injector device such as those known in the art (e.g., U.S. Pat. Nos. 5,431,654, 5,893,488, 5,638,997, 6,217,581, and 7,008,433) incorporated by reference for description of cement injectors but not to limit the terms used herein. For example, cement injectors are commercially available (e.g., Cook Medical).

Access Member and Access Member Handle Having a Side Aperture for Insertion of Inner Member

Other embodiments of the present invention recognize that in some situations, there may be a need to reduce the clearance (e.g., distance D₁ and D₂) (see FIG. 2) required for insertion of an inner member into an access member. For example, where there is restricted clearance due to positioning of a C-arm or other imaging device, it may be necessary insert the inner member into the access member from the side of the access member. This may happen, for example, where a C-arm or other monitoring apparatus needs to be positioned close to the patient's body. Thus, additional embodiments of the present invention may also provide products and methods to access an inner body part where there is restricted clearance for insertion of an invasive device.

Thus, in certain embodiments, the present invention comprises an access member comprising a side aperture for insertion of an inner member such as a rod, plunger, or other tamping instrument into the access member, so as to reduce the clearance required for insertion of the inner member into the access member when the access member is positioned in a subject. The present invention may be embodied in a variety of ways.

In one embodiment, the side aperture is introduced as a fixture that can be attached to a substantially linear access member. For example, the side port may comprise a fixture attached to the end of the access member (e.g., an end piece). In certain embodiments, the side port may be fashioned as a handle for the access member. For example, the present invention may comprise a handle for an access member, wherein the handle comprises an aperture for insertion of an inner member into the distal end of the access member such that the inner member utilizes a curvilinear path as it travels through at least a portion of the handle. In an embodiment, the access member is configured to provide percutaneous surgical access to the body part. The access member may comprise a distal end for insertion into a body part and a proximal end for access by a user. Also, in an embodiment, the aperture for insertion of the inner member is positioned so as to reduce clearance for the inner member at the proximal end of the access member during insertion of the inner member into the access member when the access member is positioned in a subject.

The fixture that is used to provide a side access for insertion of the inner member may be used with a variety of access members. In certain embodiments, the distal end of the access member does not limit whether the fixture may be used with the access member. In certain embodiments, the access member may comprise an access member having an aperture at the distal end (e.g., surface 23 of the inner member 20 of FIG. 1; i.e., an end port) for delivery of the material to a body part. In other embodiments, the access member may comprise a side port aperture for delivery of the material to a body part.

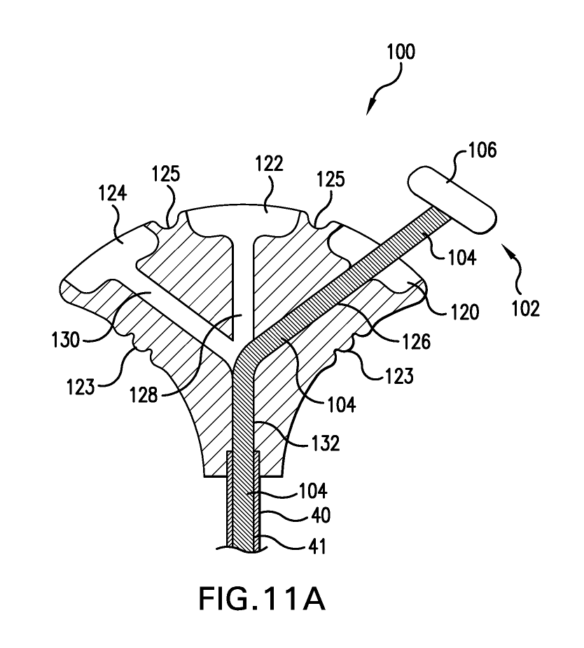

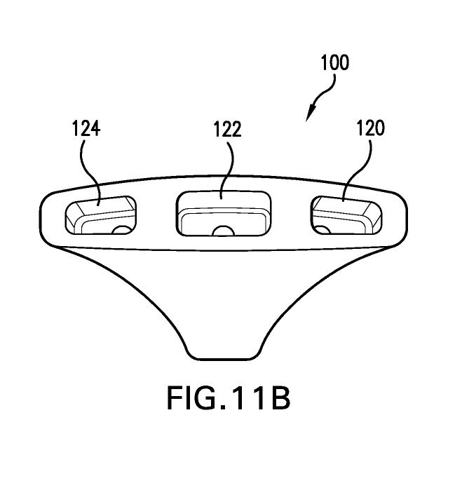

For example, as shown in FIG. 11 (Panel A and B), an access member 40 may comprise an end piece 100 comprising a plurality of ports (i.e., apertures) 120, 122, 124 that may be used to insert an inner member 102 into the access member 40. In an embodiment, the inner member comprises a handle 106 and a closed cylinder 104. The ports may connect to paths 126, 128 and 130 that are used to guide the inner cylinder 104 through the end piece to the proximal end of the access member interior 40. In an embodiment, the ports 120, 122, 124 may be shaped such that the handle of 106 of the inner member 102 can fit into the port once the inner member has been urged through the handle and the access member.

The end piece or other fixture used to add a side port may comprise only one side port. Alternatively, a plurality of side ports may be used. Where a side port 120, 124 is used, the inner cylinder 104 may be guided along a curvilinear path 126, 130 that allows for the inner member to bend as it is guided to a path 132 that is aligned with the access member. Once the inner cylinder enters the path that is aligned with the access member 40, the inner cylinder 104 may be urged through the access member using the handle 106 on the end of the inner member. The end piece 100 (or other fixture) may comprise an end port 122, as well as a side port(s) 120, 124. In one embodiment where the end port 122 and central inner path 128 is used, the inner cylinder 104 may remain in a substantially straight confirmation as it travels through the end piece 100.

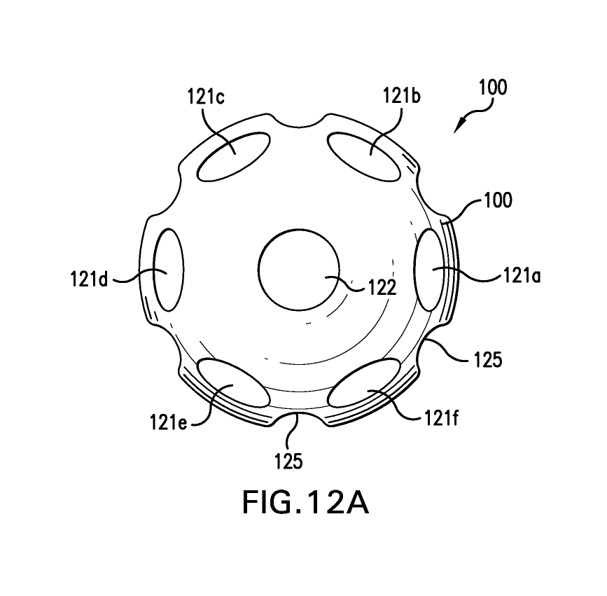

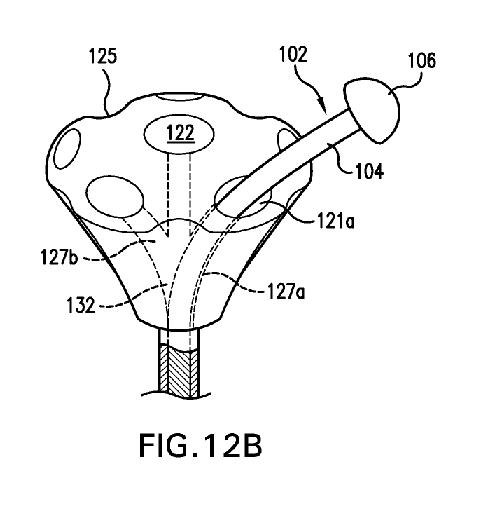

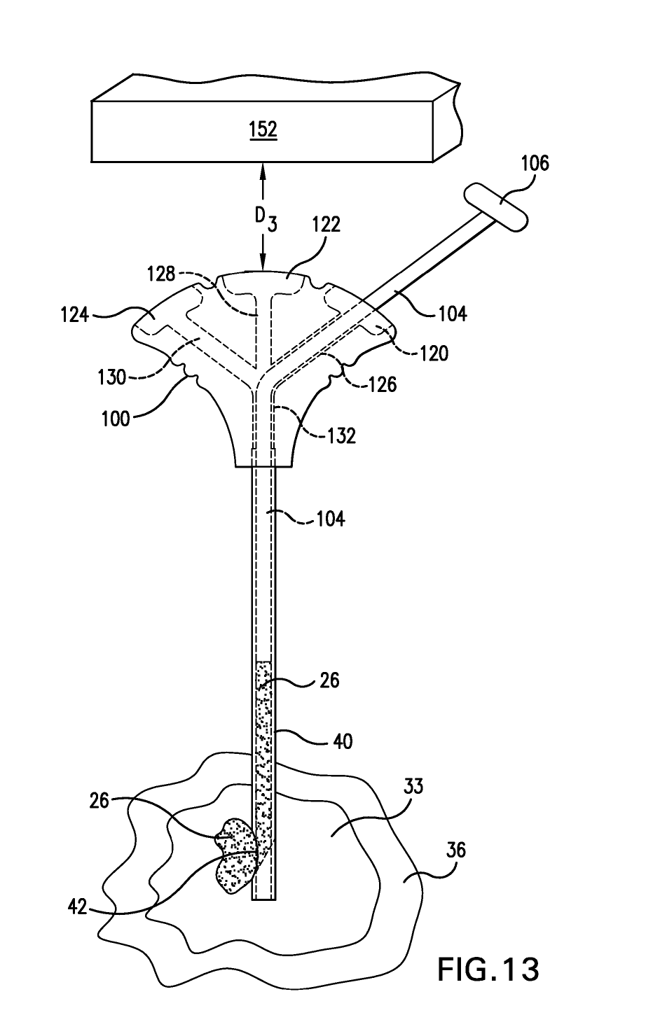

For example, in an embodiment, an end piece used with an access member may comprise three ports: one port that is at the end of the end piece (e.g., handle) to comprise a substantially straight inner path 128, 132 and two ports positioned at the side of the end piece and comprising inner paths that are curvilinear at least in part. For example, as shown in FIG. 11, panels A and B, port 120 may be connected to a curvilinear path 126 that connects to a substantially straight path 132 at the distal end of the end piece. The substantially straight path at the distal end of the end piece may then extend into the access member interior 41. The end piece may also comprise a port that provides a substantially straight inner path (e.g., 122) and one port (e.g., 124) that provides a side access. Or, the end piece may only comprise a side access port (e.g., 124). Or, the end piece may comprise two side access ports (e.g., 120, 124) but not have an end port (e.g., 122). Or, the end piece may comprise a plurality of side ports. For example, in an embodiment, a substantially circular end piece 100 with a central port 122, as well as multiple side ports 121a, 121b, 121c, 121d, 121e, 121f that are connected to curvilinear pathways e.g., 127a, 127b which connect to a substantially straight pathway 132 that connects to the proximal end of the access member may be used (FIGS. 12A and 12B). In this way, an inner member 102 may be inserted into a side port 121a from any side, thereby allowing greater flexibility in the use of the device.

The end piece may, in certain embodiments, comprise indentations 125 or other types of molding 123 to allow the user to better grip and/or manipulate the end piece. In other cases, such shaping may allow for the access member and end piece to be better aligned relative to a particular place on the patient's body (FIGS. 11 and 12).

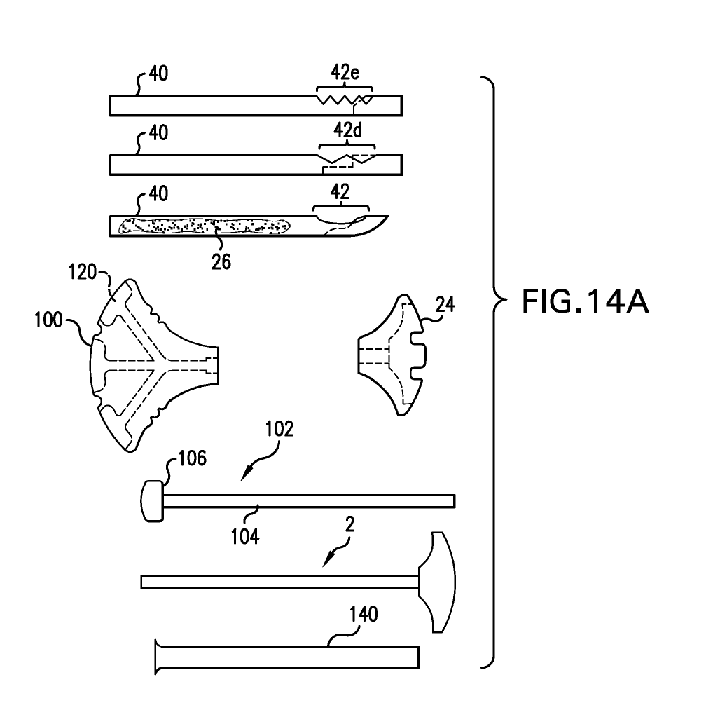

FIG. 13 shows a cross-sectional view of an end piece fashioned as a handle comprising a side port for introduction of an inner member being used to emplace a bone filler material 26 into a cavity 33 in a bone 36 where there is reduced clearance D₃ due to the presence of a C-arm or other imaging system 152. The access member shown in FIG. 13 has a side port aperture, however, an access member with an aperture at the distal end of the access member could also be used. It can be seen that, in an embodiment, where there is not sufficient clearance for the inner member 102 to be inserted into the port 122 at the end of the end piece 100, that there may be clearance for the inner member to be inserted into a port 120 at the side of the end piece. As the inner member 102 is urged through the port 120 and inner paths 126 and 132, the distal end of the inner member will push the material 26 through the access member 40 to exit the access member at side port 42.

The fixture or end piece (e.g., handle) comprising a side aperture for insertion of an inner member into the access member may be made of materials used with access members as is known in the art. Thus, the fixture comprising a side aperture for the access member may be made of any material that is appropriate for use (e.g., sterilizable) with a medical apparatus or kit. The fixture comprising a side aperture may, in certain embodiments, be made of a material that is compatible with the other parts of the system. For example, the fixture may be made of metal such as aluminum, stainless surgical steel, spring steel, a nickel titanium alloy or other alloys. Or, the fixture may be made of plastic, such as polypropylene, polyurethane, polyethylene, polyethyleneteraphthalate (PET), TEFLON®, ionomer, polycarbonate or nylon. Or, the fixture may be made of silicates or liquid crystal polymers. One of ordinary skill in the art having the benefit of this disclosure would appreciate that other materials, including those that are well-known to one in the art, may be applied to configure the side port fixture described herein.

The inner member to be used with an access member that has a fixture (e.g., handle) having a side aperture for insertion of the inner member may be an inner member such as a rod, stylet, plunger or tamping instrument of the prior art. For example, a rod, plunger, or stylet having a diameter slightly smaller than the internal diameter of the access member may be used. For use with an end piece having a side port, 120, 124 (see e.g., FIG. 13), the inner cylinder 104 may be flexible as discussed in more detail below. Materials used for the inner member may be any material that is appropriate for use within a human or animal body. Such materials may include metal such as aluminum, stainless surgical steel, spring steel, a nickel titanium alloy or other alloys. Or, the inner member may be made of plastic, such as polypropylene, polyurethane, polyethylene, polyethyleneteraphthalate (PET), TEFLON®, ionomer, polycarbonate or nylon. Or, the inner member may be made of silicates or liquid crystal polymers. In certain embodiments, the inner member may be coated with any appropriate medical grade coating including an anti-infective, an anti-coagulant, a release coating, and/or a slipping agent.

In other embodiments, the inner member may comprise a flexible material that has structural or shape “memory.” Additionally or alternatively, the inner member may comprise a linear series of substantially rigid pieces (e.g., cylinders) connected by flexible connectors that may be push through the access member but that may be bent away from any obstructions such as a C-arm or other imaging device.

In an embodiment, a shape memory material such as nitinol may be used for the inner member. As is known in the art, a shape memory material may be urged from a first shape to a second shape by the application of external energy, but when the external energy is removed, the material will resume its original shape without loss of strength or internal structure. For example, one can bend a straight wire that is made of a shape memory alloy, and upon removing the force required to bend the wire, the wire will resume its straight conformation. As is known in the art, such shape memory materials are commercially available in various compositions, conformations, surface finishes, transformation temperatures, and the like, which can be selected to optimize the performance characteristics required. Nitinol is a commonly used shape memory alloy containing almost equal parts of titanium and nickel. Nitinol may, in certain embodiments, recover from significantly greater deformation compared to most other shape memory alloys.

The material may further comprise a temperature-sensitive shape memory material such that exposure of the connector to the heat of the subject's body may result in the inner member being able to assume a second conformation different than a first conformation. For example, nitinol is a commonly used biomaterial with thermal shape memory properties. An inner member made from a temperature-sensitive shape memory alloy can be deformed (e.g., bent) to a shape suitable for insertion into an access member under conditions of limited clearance, with a thermally-induced reversal of the deformation (e.g., from bent to straight) when the inner member is threaded though the access member. The applied heat can be from the surrounding tissue, or may be externally applied. Temperature-sensitive shape memory alloys are available in a wide range of transformation temperatures appropriate for the clinical setting, including those alloys (such as nitinol) that exhibit a transformation temperature at body temperature.

Systems and Kits

In other embodiments, the products of the present invention may comprise a system or a surgical or medical kit. The system or kit may be suitable for medical or veterinary use, as for example, for emplacement of a material in a human, or an animal. For example, in an embodiment, the present invention may comprise a system comprising a device for delivering a material to a predetermined location in a subject. In another embodiment, the present invention may comprise a surgical or medical kit comprising a device for delivering a material to a predetermined location in a subject. In various embodiments of the systems and kits of the present invention, each of the embodiments of each of the products described herein may be used.

In an embodiment, the system or kit comprises components that reduces the tendency of the material extruded from the side port to adhere to the material remaining in the access member and/or reduces the tendency of the access member to become embedded in the material delivered to the body part. Also, in certain embodiments, the systems and kits of the present invention reduce the amount of force needed to remove the access member from the material that has been delivered to the body part.

For example, in one embodiment, the system or kit may comprise a product comprising an access member configured to provide percutaneous surgical access to a body part in a subject. The access member may comprise a distal end for insertion into a body part and a proximal end for access by a user. In certain embodiments, the product used in the system or kit may comprise a side port aperture positioned near the distal end of the access member for extruding a material to a body part within a subject, wherein the access member comprises a variation in the internal diameter adjacent to the side port aperture. In an embodiment, the variation in the internal volume adjacent to the side port reduces the tendency of the material extruded from the side port to adhere to the material remaining in the access member and/or reduces the tendency of the access member to become embedded in the material delivered to the body part. Also, in certain embodiments, the variation in internal diameter of the access member of the systems and kits of the present invention reduce the amount of force needed to remove the access member from the material that has been delivered to the body part. In this way, upon delivery of at least a portion of the material contained within the access member to the body part via the side port aperture, the material in the body part displays reduced adhesion to the access member and any material remaining within the access member upon retraction of the access member from the body part.

The access members described herein may be used in the systems and kits of the present invention. Thus, the access member may comprise a coating and/or a tapered distal end as described herein. Also, the portion of the access member adjacent to the side port aperture, and which provides a variation in the internal volume adjacent to the side port aperture (e.g., a ramp or the like) may be coated with plastic or other material to reduce adherence of the material to that portion of the access member as described above.

In other embodiments, the systems or kits of the present invention may comprise a product comprising a removable structure (e.g., an insert) for use with an access member having a side port for extruding a material to a predetermined location within a subject. In certain embodiments, the insert may be emplaced in the internal volume of an access member. In certain embodiments, the access member may be configured to provide percutaneous surgical access to a body part in a subject, with a distal end for insertion into a body part and a proximal end for access by a user, and a side port aperture positioned at or near the distal end of the access member for extruding a material to a body part within the subject. In an embodiment, the structure is designed such that insertion of the structure into the access member provides a variation in the internal diameter adjacent to the side port aperture.

In yet other embodiments, the systems or kits of the present invention may comprise an access member comprising a side aperture for insertion of an inner member such as a rod, plunger, or other tamping instrument into the access member, so as to reduce the clearance required for insertion of the inner member into the access member when the access member is positioned in a subject. In an embodiment, the aperture for insertion of the inner member is positioned so as to reduce clearance for the inner member at the proximal end of the access member during insertion of the inner member into the access member when the access member is positioned in a subject. In an embodiment, the side aperture is introduced as a fixture that can be attached to the proximal end of a substantially linear access member. For example, the side port may comprise a fixture attached to the end of the access member (e.g., an end piece). In certain embodiments, the side port may be fashioned as a handle for the access member. Thus certain embodiments of the systems or kits of the present invention may comprise a handle for an access member, wherein the handle comprises an aperture for insertion of an inner member into the distal end of the access member such that the inner member utilizes a curvilinear path as it travels through at least a portion of the handle.

In an embodiment, the access member is configured to provide percutaneous surgical access to the body part. The access member may comprise a distal end for insertion into a body part and a proximal end for access by a user. As described herein, the use of a endpiece fixture for insertion of the inner member into the access member is not limited by the nature of the exit port at the distal end. Thus, the access member may comprise either a side port for extrusion of the material into a body part, or an end port for extrusion of the material into a body part.

The systems and kits of the present invention may, in other embodiments, include aliquots of the material being delivered to the internal body part of interest. Materials such as bone fillers and/or bone cements described herein may be included as part of the systems or kits of the present invention.

The access member may comprise a variety of means to reduce the adherence of the material that has already been extruded to the material that remains within the access member, or to reduce the tendency of the access member to become embedded in the material. In an embodiment, the access member comprises a variation in the internal diameter of the access member adjacent to at least a portion of the side port. In certain embodiments, the variation may comprise a reduction in the inner volume or diameter for a portion of the access member. In one embodiment, the variation in the internal diameter adjacent to the side port aperture comprises a reduction in the internal diameter at the distal end of the side port aperture as compared to the proximal end of the side port aperture. For example, the variation may reduce the diameter of the access member at the distal end of the side port such that the cross-section of the material as it exits the side port is smaller at the distal end than at the proximal end.

The variation in the internal diameter adjacent to the side port aperture may comprise a variety of shapes. For example, in certain embodiments, the variation in the internal diameter adjacent to the side port aperture comprises at least one of a straight ramp, a convex ramp, a compound convex ramp, a concave ramp, a compound concave ramp, a step, an angled step, a reverse angled step, a plurality of surfaces comprising at least one convex surface and one concave surface. For example, in certain embodiments, the variation in the internal diameter adjacent to the side port aperture may comprise a plurality of steps, a wave or plurality of waves, or combinations thereof. Or, similar shapes may be used.

The variation of the internal diameter adjacent to the side port may comprise a built-in feature of the access member. Alternatively or additionally, the discontinuity within the access member may comprise an accessory feature, such as a plug, insert or other fixture that can be inserted into the substantially hollow end of an access member. For example, in an embodiment, the insert provides a variation in the internal diameter of the access member adjacent to at least a portion of the side port. In certain embodiments, the variation may comprise a reduction in the inner volume or diameter for a portion of the access member. In one embodiment, the variation in the internal diameter adjacent to the side port aperture comprises a reduction in the internal diameter at the distal end of the side port aperture as compared to the proximal end of the side port aperture.

The structure or insert may be fixedly attached to the access member, or may be removably attached to the access member. The structure may thus fit within the access member to reduce adhesion of the material being delivered by access member to the material that remains in the access member, and/or to reduce the tendency of the access member to become embedded in the material delivered to the body part.

The structure or insert for positioning adjacent to the side port aperture may comprise a variety of shapes. For example, in certain embodiments, the structure may comprise at least one of a convex ramp, a compound convex ramp, a concave ramp, a compound concave ramp, a step, an angled step, a reverse angled step, a plurality of surfaces comprising at least one convex surface and one concave surface. The structure may also comprise, in alternate embodiments a plurality of steps, a wave or plurality of waves, or combinations thereof. Or, similar shapes may be used.

For example, in an embodiment, the insertable structure may comprises an angled step, such that positioning the angled step adjacent to the side port reduces the internal volume of the access member at the distal end of the side port. Alternatively, the insertable structure may comprise at least one vertical step, such that positioning the at least one step adjacent to the side port reduces the internal volume of the access member at the distal end of the side port.

In other embodiments of the systems and kits of the present invention, the aperture used to form the side port for the access member may provide a means to reduce adherence of the material that has been delivered to the body part to the material within the access member and/or to reduce the tendency of the access member to become embedded in the material that has been delivered to the body part. For example, the side port may be shaped such that a discontinuity is introduced into the material as it exits the side port aperture. The material may be preferentially sheared or a fracture initiated at these points of discontinuity rather than in other parts of the material.

For example, the side port may be shaped such that there is a point at which the material is forced through an aperture that comprises series of points or edges such that the material may easily fracture as the access member is repositioned (e.g. twisted) away from the extruded or emplaced material. Thus, in certain embodiments, the access member of the systems and kits of the present invention comprises a discontinuity in the shape of the side port. In an embodiment, there may be at least a portion of the side port that comprises a region where the port is substantially reduced in size as compared to at least one other part of the side port. Alternatively or additionally, there may be a portion of the side port comprises a plurality of straight edges or serrated. Examples of side ports that may be used in the systems or kits of the present invention to introduce a discontinuity in a material as the material is urged through the side port include a side ports shaped as a compound curve, a tear drop, a diamond, interlaced diamonds, an oval with serrated edges, an eye, a bifurcated side port, or a serrated bifurcated side port, such as the side ports shown in FIG. 10.

The subject for which the systems and kits of the present invention may be employed may comprise an animal. For example, the subject may comprise a mammal. In one embodiment, the subject may be a human (e.g., a patient). The user of the system or kit may be a physician, veterinarian, or a health care professional (e.g., physician's assistant, nurse, or technician). In alternate embodiments, however, a user of the systems and/or kits of the present invention may be accessing a particular location in his or her own body, as for example, for periodic delivery of a therapeutic material.

The predetermined location may, in certain embodiments of the systems and kits of the present invention, comprise a body part within a living body. In an embodiment, the predetermined location may comprise a bone. In one embodiment, the predetermined location may comprise a bone interior. For example, the predetermined location may comprise a portion of a spine. Thus, in one embodiment, the access member may be sized to deliver the material to a bone interior, such as a vertebral body or disc of a spine.

For example, as described herein, due to various traumatic or pathologic conditions, such as osteoporosis, a vertebral body can experience a vertebral compression fracture (VCF). Thus, the systems and kits of the present invention may by used to repair a vertebral body lost due to a fracture, or when other degeneration occurs. The systems and kits of the present invention may also be used to repair other parts of a living or non-living organism. For example, in certain embodiments, the kits and systems of the present invention can be deployed in other bone types and within or adjacent other tissue types, such as in a vertebral disc, an arm bone, a leg bone, a knee joint, or the like.

The access member used with the systems and kits of the present invention may provide a path to access a region or a body part that is located within a subject's body. The access member may be any type of device that can extend from the location of interest (e.g., a bone or an organ) to be accessible to a user of the access member. For example, the access member may be designed to extend from an internal body part in a subject to outside of the subject's body. The access member may comprise an elongated hollow member such as a hollow cylinder or a tube. Thus, in an embodiment, the tube may be designed to provide an access from outside of the living body to the internal body part. In an embodiment, the inner member and the access member are substantially cylindrical in shape. For example, the access member may comprise a cannula, such as a cannula used to deliver a material to bone or another type of body part.

In certain embodiments, the access member of the systems and kits of the present invention may be configured to provide percutaneous surgical access from outside of the subject to the predetermined location. In alternate embodiments, the percutaneous surgical access may comprise an incision ranging from about 0.05 to 8 centimeters (cm), 0.1 to 4.0 cm in diameter, or from about 0.2 to 2.0 cm in diameter, or from about 0.25 to about 1 cm in diameter. Or ranges within these ranges may be used. Thus, in alternate embodiments, the percutaneous surgical access may comprise an incision that is less than 4 cm in diameter, or less than 2 cm in diameter, or less than 1 cm in diameter. In one example embodiment, the percutaneous surgical access may comprise an incision of about 1 cm in diameter. For example, in a typical percutaneous surgical repair of a spine, a cannula may establish a percutaneous path along its elongated axis to a vertebral body of one of the several vertebrae.

In an embodiment, the kit may comprise a container for holding each of the parts under sterile conditions, or for transporting the parts from a first site to a second site. The kit may, in some embodiments, comprise a tray for holding the various parts in a secure position during sterilization and/or transport. In an embodiment, the parts of the kit are arranged in an organized layout to facilitate use of the access member and other components of the kit for delivery of material to an internal body part. Also, labels identifying various parts of the kit may be included.

In an embodiment, the kit may comprise an inner seal, such as an inner wrap that may be sealed by heat or vacuum, to prevent the components of the kit from being exposed to the outside environment. The inner seal may comprise a conventional peal-away seal to provide quick access to the components of the kit. Also, in an embodiment, the kit may include an outer wrap, also sealed by heat or the like, to enclose the inner wrap. Like the inner seal, the outer seal may comprise a conventional peal-away seal to provide quick access to the components of the kit. Use of an outer wrap may allow the kit to be prepared for imminent use by removing the outer wrap while leaving the inner wrap in place to ensure sterility of the kit components. The kit may also comprise a case to protect the components of the system from physical damage. In an embodiment, the outer wrap may be made of materials commonly used in the art such as polyethylene and MYLAR™, to allow for visualization of the components in the kit. The inner wrap may be made of materials such as TYVEK™ (DUPONT®), that is permeable to ethylene oxide (ETO) sterilizing gas. Sterilization may be by heat, pressure and/or sterilization gas as is known in the art. Also, the kit may include directions for use by a physician or other trained personnel.

Embodiments of the systems and/or kits of the present invention may further comprise a material to be delivered to the internal body part. In an embodiment, the material to be delivered to the body part may be emplaced within at least a portion of at least one access member. For example, where the system is being used for bone repair, a tube may be loaded with a bone filler material or a bone cement. In an embodiment, the bone filler material may comprise a mixture containing calcium, hydroxyl apatite, and a polymer. Also, in certain embodiments, the bone filler may comprise ceramic granules or other filler material.

In yet other embodiments, the material may comprise an autograft or allograft bone graft tissue (see e.g., Dick, Archives of Orthopaedic and Traumatic Surgery (1986), 105: 235-238; or Bhan et al, International Orthopaedics (SICOT) (1993) 17: 310-312). The bone graft tissue can be obtained using a Bone Graft Harvester, which is commercially available from SpineTech. Alternatively, the material may also comprise a granular bone material harvested from coral, e.g., PROOSTEON™ calcium carbonate granules, available from Interpore. The granules may be loaded into the access member using a funnel or other loading means. The material for delivery to a bone can also comprise demineralized bone matrix suspended in glycerol (e.g., GRAFTON™ allograft material available from Osteotech), or SRS™ calcium phosphate cement available from Novian. The material for delivery to a bone can also be in sheet form, e.g., COLLAGRAFT™ material made from calcium carbonate powder and collagen from bovine bone. In an embodiment, the sheet may be rolled into a tube and loaded by hand into the access member.

In an embodiment, the material for emplacement in the body part comprises a bone cement. Example bone cements that may be used include, but are not limited to polymethyl methacrylate (PMMA) based-bone cement, resorbable bone cement, bone cement that includes osteo-inductive materials, and bone cement with a relatively fast cure time (e.g., less that 25, 20, 15, or 10 minutes). Example bone cements that may be used include KYPHX® HV-R bone cement (dough time of 8 to 16 minutes; set time of about 19.8 to 21.3 min), and KYPHX® QV-R bone cement (dough time of about 4.5 minutes to 9 minutes; set time of about 12.9 to 13.4 minutes), commercially available from Kyphon, Inc. In an embodiment, an injectable calcium-phosphate cement (e.g., Weitao et al., J. Postgrad. Med., 2007, 53:34-38), or hydroxyapatite composite materials (e.g., hydroxyapatite/ceramic composites and/or hydroxyapatite/polymer composites) may be used.

The system and/or kits of the present invention may comprise one, or a plurality, of access members (e.g., cannulas) that are loaded with material that is to be delivered to a predetermined location in a subject, such as a body part or region. Thus, in one embodiment, after the material in one of the access members has been dispensed to the body part of interest, the first access member and inner member (e.g., rod or plunger) may be removed and an additional access member filled with material to be emplaced may be positioned for delivery of a second aliquot of the material. At this point, an additional inner member may be inserted into the newly placed access member. Or, the inner member used to deliver the first aliquot of the material may be removed from the first (e.g., substantially spent) access member and used to deliver a second aliquot of material from the second access member.

The access members (e.g., tubes or cannulas), and inner members, may vary in size depending upon the body part to be accessed. In one embodiment, the systems and/or kits of the present invention may comprise a bone filler device. The bone filler device may range in size depending upon the bone to be repaired. For example, the access member and inner members may be sized to fit pre-existing bone filler devices such as the KYPHX® EXPRESS™ bone filler device that is commercially available from Kyphon, Inc. For example, to repair a single thoracic vertebra may require up to about 12 cubic centimeters (cc) of bone filler material. Using a cannula that is about 8-12 inches (203 mm) long and about 0.137 inches (3.5 mm) wide may require about 6 to 8 cannulas of bone filler material. More or less material may also be delivered depending upon the size of the cavity to be filled. Where a cavity is created by drilling a hole in a bone and compacting any bony material with an expandable device such as a balloon, the amount of material required for a specific application may be determined by monitoring the inflation volume of the balloon.

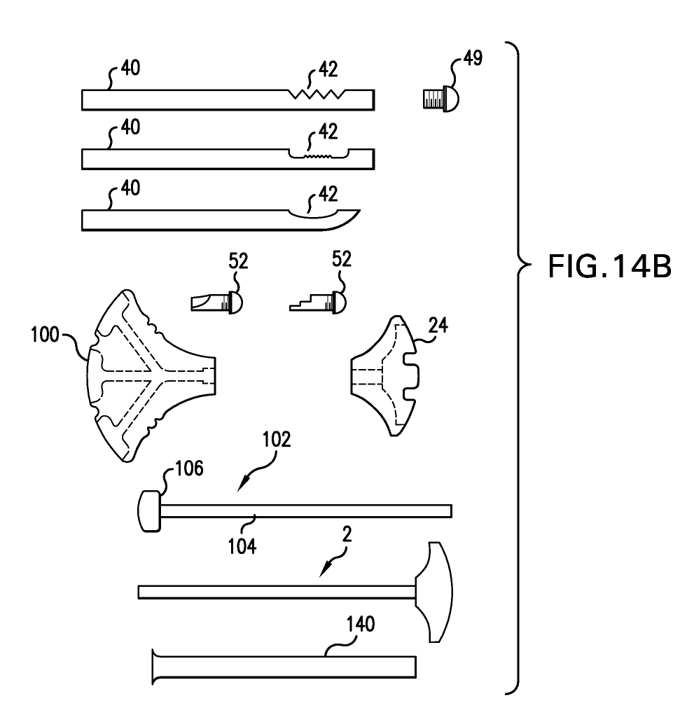

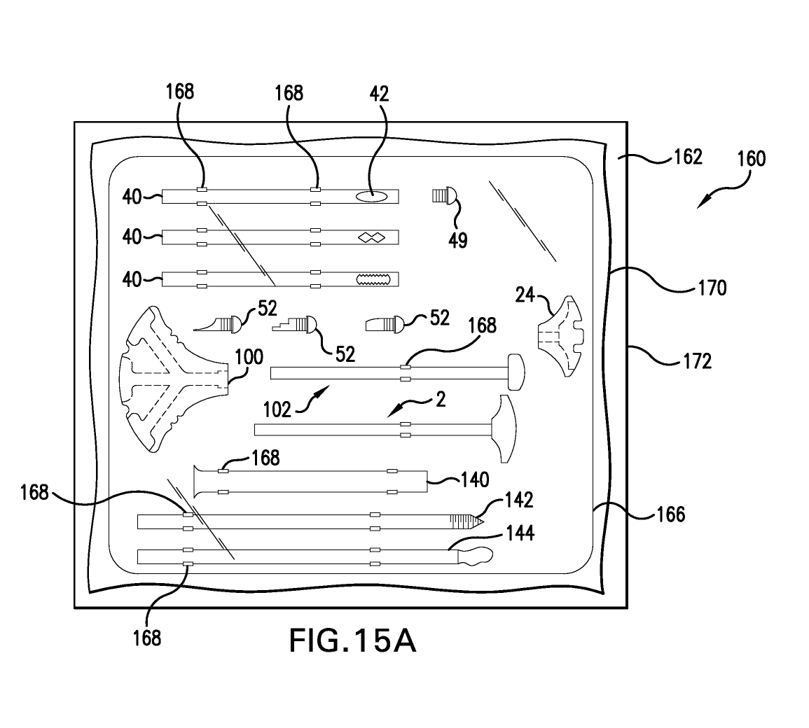

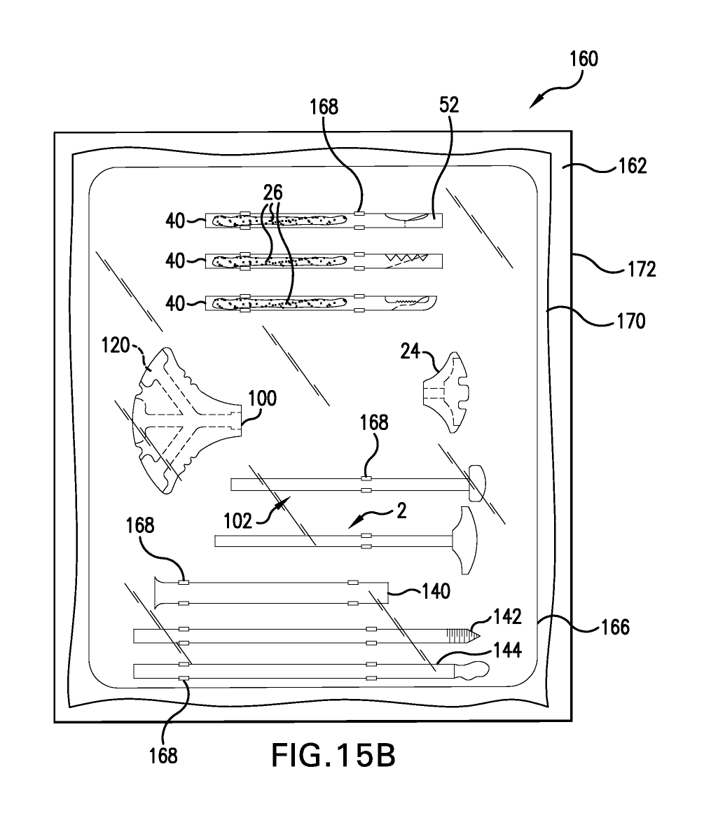

Embodiments of a system of the present invention are shown as FIG. 14, panels A and B. Although only certain embodiments of the invention are displayed, each of the embodiments described herein may be used in the kits and systems of the present invention.

Thus, in some embodiments, the system may comprise one or more inner members 102 and one or more access members 40. The access members may be substantially straight, or may be tapered throughout the length of the cannula as described in FIG. 9. Or, the access members may be tapered at the distal end. In an embodiment, the access member may comprise a side port 42 for extrusion of a material 26 to a body part of interest. Alternatively, the access member may comprise an end port for extrusion of a material 26 to a body part of interest (not shown). In an embodiment, the side port may comprise a shape to introduce at least one point of discontinuity in a material 26 as the material is extruded from the access member. For example, in an embodiment, the side port is shaped as a series of interlacing diamonds 42d (FIG. 14A). Alternatively, the side port may comprise a serrated edge 42e (FIG. 14A).

In an embodiment, the access member 40 may be preloaded with a material 26 to be emplaced in a body part of interest (e.g., FIG. 14A). Alternatively, the material may be loaded into the access member during use.

The inner member(s) 2 or 102 may have a diameter that is less than the internal diameter of the access member(s) 40 and a length less than the length of the access member that will be used to access the location of interest in the subject. In an embodiment, the inner member 2 or 102 may comprise an inner cylinder 4 or 104 a handle 6 or 106. The handle 106 may be fixedly attached or removably attached to the inner cylinder 104.

Also, in some embodiments, the access member 40 may comprise a handle. The handle may be fixedly attached to the access member, or the handle may be removable. In an embodiment, the handle may comprise an end port and a substantially straight inner path for insertion of the access member (e.g., 24 for use with inner member 2). Alternatively or additionally, an end piece fixture 100 comprising at least one side port 120 for use with inner member 100 may be used in the system. Or, as discussed herein, other fixtures may be used to add an injection side port to the access member.

The systems and kits may also comprise a cement injector as described herein.

In addition to the access member 40 used to deliver a material to the internal body part, the system may comprise a second member 140 comprising a means to access the body part of interest. In an embodiment, the second access member 140 may comprise a hollow cylinder or tube. The second access member may comprise a diameter that is greater than the diameter of the first access member 40 to allow an the first access member 40 to be inserted into the second member 140 and threaded to the body part of interest. For example, the system may include a drill bit or other cutting tool for cutting into a bone requiring repair. Once the interior of the bone has been accessed, a balloon catheter may be inserted into the second access member 140 and threaded to the site of the bone requiring repair. Inflation of the balloon may then be used to compact any deteriorated bone tissue, and to create a cavity in the bone. Thus, alternate embodiments the system may also comprise a cutting tool (not shown) and a balloon catheter (not shown) similar to those in the art used for the repair of bone.

Embodiments of the systems may comprise an access member having a portion for introducing reduced adherence of the material that is extruded from the access member to the material remaining in the access member, or to reduce the tendency of the access member to become embedded in the material delivered to the body part. In an embodiment, the structure is built into the access member (FIG. 14A). Alternatively, the structure is a separate piece 52 that may be inserted into the access member (FIG. 14B).

Embodiments of a kit 160 of the present invention are shown as FIG. 15, panels A and B. In an embodiment, the kit may comprise a container 162 for holding each of the parts under sterile conditions, or for transporting the parts from a first site to a second site. The kit may comprise one or a plurality of inner members 2 or 102 and one or a plurality of access members 40. The access members may be substantially straight, or may be tapered as described in FIG. 9.

The access members may be substantially straight, or may be tapered throughout the length of the cannula as described in FIG. 9. Or, the access members may be tapered at the distal end. In an embodiment, the access member may comprise a side port 42 for extrusion of a material 26 to a body part of interest. Alternatively, the access member may comprise an end port for extrusion of a material 26 to a body part of interest (not shown). In an embodiment, the side port may comprise a shape to introduce at least one point of discontinuity in a material 26 as the material is extruded from the access member. For example, in an embodiment, the side port is shaped as a series of interlacing diamonds. Alternatively, the side port may comprise a serrated edge (FIG. 15).

In an embodiment, the material may be loaded into the access member during use (FIG. 15A). Alternatively, the access member 40 may be preloaded with a material to be emplaced 26 in a body part of interest (FIG. 15B).

The inner member(s) 2 or 102 may have a diameter that is less than the internal diameter of the access member(s) 40, and a length less than the length of the access member 40 that will be used to access the location of interest in the subject. In an embodiment, the inner member 2 or 102 may comprise an inner cylinder 4 or 104 a handle 6 or 106 (FIGS. 1 and 15). The handle 106 may be fixedly attached or removably attached to the inner cylinder 104.

Also, in some embodiments, the access member 40 may comprise an end piece or handle. The handle may be fixedly attached to the access member, or the handle may be removable. In an embodiment, the handle may comprise an end port and a substantially straight inner path for insertion of the access member such as handles used with bone filler devices of the prior art (e.g., 24). Alternatively or additionally, an end piece 100 comprising at least one side port 120 may be used in the kits of the present invention. Or, as discussed herein, other fixtures may be used to add an injection side port to the access member.

Embodiments of the kits may comprise an access member having a structure for introducing reduced adherence of the material that is extruded from the access member to the material remaining in the access member, or to reduce the tendency of the access member to become embedded in the material. In an embodiment, the structure is a separate piece 52 that may be inserted into the access member (FIG. 15A). Alternatively, the structure is built into the access member (FIG. 15B). In yet another embodiment, a tapered access member, such as the access members shown in FIG. 9 may be used. Also, in certain embodiments, the distal end of the access member may be coated with a substance that has reduce adhesion to the material being delivered.

Also, an outer access member 140, drill bit 142, balloon catheter 144, and other components (e.g., cement injector) may be included. The kit may comprise a tray 166 comprising clips 168 or other fastening means for holding the various parts in a secure position during sterilization and/or transport. In an embodiment, the parts of the kit are arranged in an organized layout to facilitate use of the access member for delivery of material to an internal body part. Also, labels identifying various parts of the kit may be included. The kit and components therein (e.g. access members having a discontinuity in the internal volume next to a side port or a discontinuity in the shape of the side port) may, in certain embodiments be used with a cement injector.

In an embodiment, the kit may comprise an inner wrap 170, that may be sealed by heat or vacuum to prevent the components of the kit from being exposed to the outside environment. Also, in an embodiment, the kit may include an outer wrap (not shown) and/or a case 162 to protect the components of the system from physical damage. The inner/outer wraps may be made of wrap materials commonly used in the art such as polyethylene, TYVEK™, or MYLAR™, to allow for visualization of the components in the kit and or sterilization using a sterilizing gas. Sterilization may be by heat, pressure and/or sterilization gas as is known in the art. Also, the kit may include directions for use by a physician or other trained personnel.

Methods for Delivery of a Material to a Body Part of Interest

Embodiments of the present invention may also comprise methods for delivery of a material to an internal body part. In certain embodiments, the present invention comprises methods for delivery of a material to a body part in a subject using an access member configured to provide percutaneous surgical access to the body part.

In an embodiment, the method reduces the tendency of an access member to become embedded in a material delivered to a body part. For, example, the method may reduce adhesion of the material delivered to the body part to material that remains in the access member. The method may comprise the steps of inserting the access member in the subject such that the distal end of the access member is positioned within the body part, or juxtaposed adjacent to an aperture in the body part, wherein at least a portion of the internal volume of the access member comprises at least a portion of the material to be delivered to the body part; and urging an inner member at least partially through the access member to deliver at least a portion of the material to the body part wherein the access member comprises a side port aperture positioned near the distal end of the access member for extruding the material to the body part, and wherein the access member comprises a variation in the internal diameter adjacent to the side port aperture. In an embodiment, the variation in the internal volume adjacent to the side port reduces the tendency of the material extruded from the side port to adhere to the material remaining in the access member. In certain embodiments, the methods of the present invention reduce the amount of force needed to remove the access member from the material that has been delivered to the body part. In this way, upon delivery of at least a portion of the material contained within the access member to the body part via the side port aperture, the material in the body part displays reduced adhesion to the access member and any material remaining within the access member upon retraction of the access member from the body part. In certain embodiments, the material for delivery to the body part is a bone filler or cement.

The access member may comprise a variety of means by which the adherence of the material that has already been extruded to the material that remains within the access member, or to reduce the tendency of the access member to become embedded in the material delivered to the body part.

In one embodiment, the access member may comprise a variation in the internal diameter of the access member adjacent to at least a portion of the side port. In certain embodiments, the variation may comprise a reduction in the inner volume or diameter for a portion of the access member. In one embodiment, the variation in the internal diameter adjacent to the side port aperture comprises a reduction in the internal diameter at the distal end of the side port aperture as compared to the proximal end of the side port aperture. For example, the variation may reduce the diameter of the access member at the distal end of the side port such that the cross-section of the material as it exits the side port is smaller at the distal end than at the proximal end.

The variation in the internal diameter adjacent to the side port aperture may comprise a variety of shapes. For example, in certain embodiments, the variation in the internal diameter adjacent to the side port aperture comprises at least one of a straight ramp, a convex ramp, a compound convex ramp, a concave ramp, a compound concave ramp, a step, an angled step, a reverse angled step, a plurality of surfaces comprising at least one convex surface and one concave surface. For example, in certain embodiments, the variation in the internal diameter adjacent to the side port aperture may comprise a plurality of steps, a wave or plurality of waves, or combinations thereof. Or, similar shapes may be used.

The variation of the internal diameter adjacent to the side port may comprise a built-in feature of the access member. Alternatively or additionally, the discontinuity within the access member may comprise an accessory feature, such as a plug or other fixture that can be inserted into a substantially hollow end of an access member. Thus, in one embodiment, the method comprises a step of inserting a structure to introduce a variation in the internal diameter adjacent to the side port aperture.

The structure that is inserted into the access member may comprise a variety of means by which the adherence of the material that has already been extruded to the material that remains within the access member, or to the access member itself, is reduced. In an embodiment, the insert provides a variation in the internal diameter of the access member adjacent to at least a portion of the side port. In certain embodiments, the variation may comprise a reduction in the inner volume or diameter for a portion of the access member. In one embodiment, the variation in the internal diameter adjacent to the side port aperture comprises a reduction in the internal diameter at the distal end of the side port aperture as compared to the proximal end of the side port aperture.

The structure for inserting into the access member and positioning adjacent to the side port aperture may comprise a variety of shapes. For example, in certain embodiments, the structure may comprise at least one of a convex ramp, a compound convex ramp, a concave ramp, a compound concave ramp, a step, an angled step, a reverse angled step, a plurality of surfaces comprising at least one convex surface and one concave surface. In certain embodiments, the variation comprises an incline from the inner wall that is about opposite the proximal end of the side port aperture to the inner wall that abuts the distal end of the side port aperture. Or similar shapes and/or combinations of such shapes may be used. The structure may also comprise, in alternate embodiments a plurality of steps, a wave or plurality of waves, or combinations thereof. Or, similar shapes may be used. For example, in an embodiment, the insertable structure may comprise an angled step, such that positioning the angled step adjacent to the side port reduces the internal volume of the access member at the distal end of the side port. Alternatively, the insertable structure may comprise at least one vertical step, such that positioning the at least one step adjacent to the side port reduces the internal volume of the access member at the distal end of the side port.

In yet another embodiment, the shape of the access member provides a means to decrease adherence of the material being delivered to a body part to the material remaining in the access member or to the access member itself. Thus, in certain embodiments of the method, the access member may comprise a reduction in the external cross-section of at least a portion of the distal end of the access member that includes the side port aperture. In an embodiment, the access member comprises a radius that exhibits a reduction in size along the length of the access member, such that the access member has a smaller radius at the distal end than at the proximal end (see e.g., FIG. 9A). Or, a portion of the distal end of the access member (e.g., adjacent to the window) may be tapered. In an embodiment, both the internal radius, and the external radius decrease from the proximal end to the distal end of the access member. Also, in an embodiment, the access member may comprise an internal radius and an external radius that are significantly narrowed adjacent to, or over the length of, the opening.

The external surface of the access member used in the methods of the present invention may be curved so as to aid in removal of the access member from the material deposited in the body part. For example, the access member may comprise a concave outer surface that is forms the back side of the side port aperture. In certain embodiments, the outer surface may mimic a concave curvature of inner surface of the access member that is adjacent to the side port aperture. In an embodiment, the curved outer surface extends to a narrow tip at the end of the access member thereby reducing the tendency of the access member to become embedded in the material that has been extruded into the body part. Also, the curved tip may facilitate extricating the access member from the material as there may be an area adjacent to the curved surface that is not embedded in the material, but that comprises a small void in the material (see e.g., FIG. 9A).

Or, the access member may comprise a tapered distal end, but have a substantially flat surface that forms the back side of the side port aperture (FIG. 9B). In an embodiment, the tapered outer surface extends to a narrow tip at the end of the access member thereby reducing the tendency of the access member to become embedded in the material that has been extruded into the body part. Also, the tapered tip may facilitate extricating the access member from the material as there may be an area adjacent to the tapered surface that is not embedded in the material, but that comprises a small void in the material.

In another embodiment of the method, the access member may comprise an indentation along the outer surface which provides a convex curvature to the internal radius of the access member. In this embodiment, a material will be directed out of the access member and away from the back side of the opening. Thus, there may be a reduced tendency for the material to cure around the back side of the access member. Additionally, the tapered shape of the access member may facilitate creating a discontinuity or void in the material being delivered to the body part.

In certain embodiments, the access member may be coated with a material to help reduce adhesion of the access member to the material being extruded from the access member. In an embodiment, at least a portion of the distal end of the access member that includes the side port aperture is coated with a plastic such as TEFLON®, or similar plastics, or a slipping agent or release agent as described herein.

In other embodiments of the method, the side port aperture itself may be used to provide a means by which to induce reduced adherence of the material being delivered to a body part to either the access member or any material remaining within the access member. In certain embodiments, the side port aperture may comprise a shape such that upon delivery of at least a portion of the material contained within the access member to the body part via the side port aperture, the shape of the side port aperture reduces the tendency of the material extruded from the side port to adhere to the material remaining in the access member. Thus, in certain embodiments, the side port aperture is shaped such that the adhesive material in the body part displays less adhesion to the access member and any material remaining within the access member upon retraction of the access member from the body part than when a round or oval side port aperture is used. For example, the side port may be shaped such that a discontinuity is introduced into the material at certain points as the material exits the access member. In one embodiment, the side port may be shaped such that the material is sheared at least in part at certain points as it exits the access member. Or, the side port may be shaped such that a fracture point is induced in the material at certain points as the access member is repositioned relative to the material that has been delivered to the body part. Or, the side port may be shaped such that a fracture point is induced in the material at certain points as the material exits the access member. Or, the side port may be shaped such that there is a point at which the material is forced through an aperture that comprises series of points or edges. In this way, the material may easily fracture as the access member is twisted away from the extruded or emplaced material.

Thus in certain embodiments, the access member may comprise a discontinuity in the shape of the side port. In certain embodiments, the side port comprises at least one of a serrated edge, a discontinuity in the shape of aperture such that the width of at least one portion of the aperture is substantially reduced in size as compared to the width at another portion of the aperture, or a division of the aperture into a plurality of apertures. Alternatively or additionally, there may be a portion of the side port comprises a plurality of serrated or straight edges.

The method may comprise the step of intentionally using the side port aperture to sever the material that has been delivered to the body part from material that remains in the access member. For example, in certain embodiments, the present invention may comprise a method for delivery of a material to a body part in a subject comprising the steps of: (a) positioning an access member comprising a side port aperture for emplacing a material in a body part, wherein the access member comprises a material to be emplaced in the body part; (b) delivering at least a portion of the material to the body part via the side port aperture; (c) intentionally leaving the access member in the body part until the material has hardened or cured; and (d) removing or repositioning the access member in a manner such that the side port is used to sever material that remains in the distal portion of the access member from material that has been emplaced in the body part.

For example, in one embodiment, the method may comprise the steps of positioning an access member in the body part, delivering at least a portion of the material to the body part, intentionally leaving the access member in the body part until the material has hardened or cured, and removing or repositioning the access member in a manner such that the side port is used to sever the material that remains in the distal portion of the access member from the material immediately outside of the side port aperture. In an embodiment, the access member is repositioned to sever the material. For example, in alternate embodiments, the access member may be twisted, rotated, jiggled or pulled in the proximal direction as a means to sever the cement. Also, in certain embodiments, the material is a bone cement or a bone filler. Also, in certain embodiments, the access member is configured for percutaneous access.

As discussed herein, a variety of side ports may be used to introduce a discontinuity in a material as the material is urged through the side port so as to introduce reduced adherence of the access member to the extruded material. For example, the access member may comprise a side port shaped as a compound curve, or a tear drop, or a diamond, or two (or more) interlaced diamonds, or an oval with serrated edges, or an eye, or a bifurcated side port, or serrated bifurcated side port. Or other shapes that comprise a means to introduce a discontinuity in the material being delivered may be used.

For example, in one embodiment, the method may comprise a method for delivery of a material to a predetermined location in a subject comprising the steps of: inserting an end of inner member into the end of an access member comprising at least a portion of the material to be delivered, the access member comprising a side port for extruding a material to a predetermined location within a subject, wherein at least a portion of the access member induces reduced adhesion of the material that is delivered material remaining in the access member and/or reduced tendency of the access member to become embedded in the material. In an embodiment, the method may further comprise repositioning the access member to initiate a discontinuity in the material being delivered. For example, once the material has been extruded from the access member, the access member may be twisted or otherwise repositioned to sever the material that remains in the access member away from the material that has been emplaced.

The access member may be loaded with an amount of the material for delivery to the predetermined location as is required. In alternate embodiments, the access member may comprise an amount of material that is less than, about the same as, or greater than, the amount of material that is ultimately to be delivered to the location of interest. Thus, in an embodiment, the method may further comprise the step of loading the access member with at least a portion of the material to be delivered to the predetermined location in a subject. As described above, the access member may provide access to the predetermined location for person performing the method. Thus, the method may further comprise the step of positioning the access member such that one end of the access member is located at the predetermined location and the other end of the access member is accessible to a user.

For example, in an embodiment, the method may comprise delivering a bone cement or filler material to a bone requiring repair. Thus, in one example embodiment, the method may comprise the steps of: loading an access member comprising a side port with at least a portion of a material to be delivered to a predetermined location in a subject, wherein the access member comprises a side port aperture positioned near the distal end of the access member for extruding the material to the body part, and wherein the access member comprises a variation in the internal diameter adjacent to the side port aperture, and wherein the variation in the internal volume adjacent to the side port aperture reduces the tendency of the material extruded from the side port to adhere to the material remaining in the access member. The method may also comprise the steps of positioning the access member such that one end of the access member is located at the predetermined location and the other end of the access member is accessible to a user; inserting an end of an inner member into the end of the access end of the member that is farthest from the predetermined location in the subject; urging the inner member towards the end of the access member positioned at the predetermined location in the subject to thereby urge the material in the access member towards the predetermined location; and optionally, repositioning the access member so as to introduce a fracture or loss of cohesiveness in the material being emplaced.

In some cases, the method may further include the steps of removing the first inner member and inserting an end of an additional inner member into the proximal end of the access member and urging the additional inner member towards the distal end of the access member to thereby urge the material in the access member towards the predetermined location. The step of inserting additional inner members and urging such inner members through the access member may be repeated until the correct amount of material is delivered to the predetermined location in the subject.

The methods of the present invention may be employed to deliver a material to a predetermined location in a subject where the subject comprises an animal. In an embodiment, the subject may be a mammal. For example, the subject may be a human (e.g., a patient).

The predetermined location may, in certain embodiments, comprise a body part within a living body. In an embodiment, the predetermined location may comprise a bone. In one embodiment, the predetermined location may comprise a bone interior. For example, the predetermined location may comprise a portion of a spine. Thus, in one embodiment, the access member may be sized to deliver the material to a bone interior, such as a vertebral body or disc of a spine.

For example, due to various traumatic or pathologic conditions, such as osteoporosis, a vertebral body can experience a vertebral compression fracture (VCF). Thus, the methods of the present invention may by used to repair a vertebral body lost due to a fracture, or when other degeneration occurs. The methods of the present invention are not, however, limited in application to vertebrae, and may be used to repair other parts of a living or non-living organism. For example, in certain embodiments, methods of the present invention can be deployed in other bone types and within or adjacent other tissue types, such as in a vertebral disc, an arm bone, a leg bone, a knee joint, or the like.

The access member used in the methods of the present invention may comprise any of the embodiments described herein. Thus, as described above, the access member may provide a path to access a region or a body part that is located within a subject's body. For example, the access member may be designed to extend from an internal body part in a subject to outside of the subject's body. The access member may comprise an elongated hollow member such as a hollow tube. In an embodiment, the inner member and the access member are substantially cylindrical in shape. Or, the access member may be other shapes (e.g., polygonal) as described herein. For example, the access member may comprise a cannula, such as a cannula used to deliver a material to bone or another type of body part.

In an embodiment, the access member used in the methods of the invention may be configured to provide percutaneous surgical access from outside of the subject to the predetermined location. In alternate embodiments, the percutaneous surgical access may comprises an incision ranging from about 0.5 to 8.0 centimeters (cm), 0.1 to 4.0 cm in diameter, or from about 0.2 to 2.0 cm in diameter, or from about 0.25 to about 1 cm in diameter. Thus, in alternate embodiments, the percutaneous surgical access may comprise an incision that is less than 4 cm in diameter, or less than 2 cm in diameter, or less than 1 cm in diameter. In one example embodiment, the percutaneous surgical access may comprise an incision of about 1 cm in diameter. For example, in a typical percutaneous surgical repair of a spine, a cannula may establish a percutaneous path along its elongated axis to a vertebral body of one of the several vertebrae.

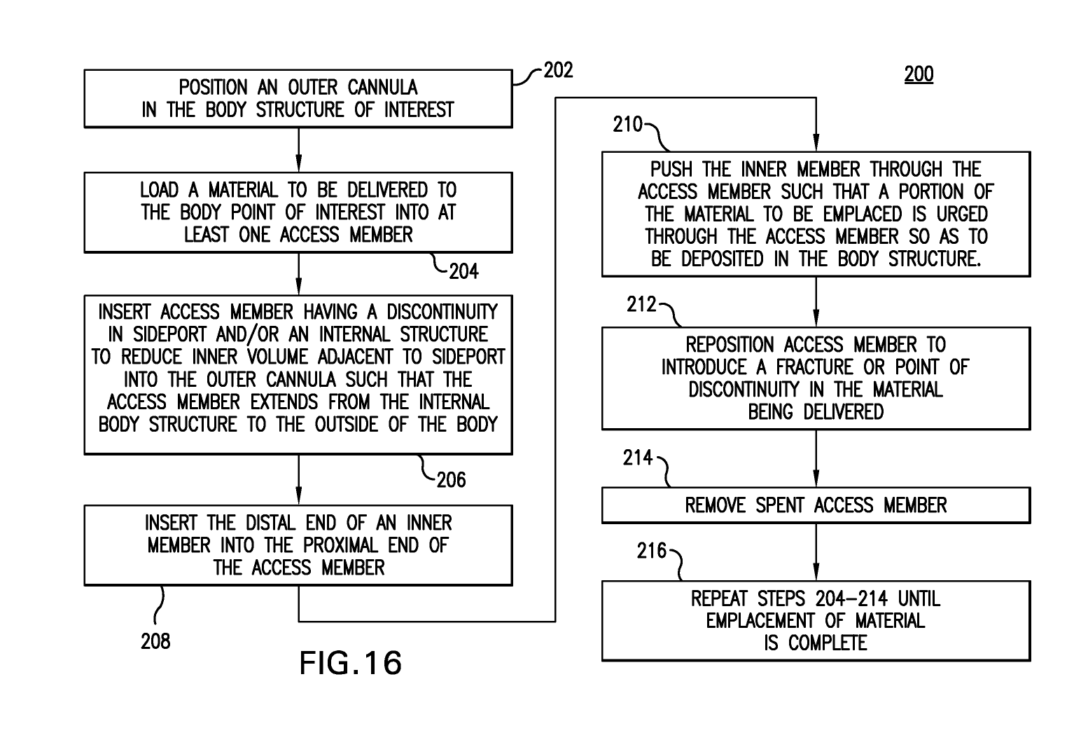

An embodiment of a method 200 of the present invention is shown in FIG. 16. For example, the method may comprise a first step 202 of positioning an outer cannula in the body part of interest. The cannula, like the access member, may be configured to provide percutaneous surgical access to a body region or body part of interest. For example, for repair of a bone, the cannula may be used to position a drill bit to cut an opening in the bone to be repaired. Also, the cannula may be used to deliver a balloon catheter to the bone to further stabilize the bone by compacting any loose tissue at the site of repair.

Next, a material to be emplaced in the body part of interest may be loaded into at least one access member 204. For example, for delivery of a bone filler material to a vertebral bone, about 6-8 access members each comprising about 1.5 cc of bone filler may be used.

At this point, one of the access members comprising the material to be emplaced may be threaded through the outer cannula to the body part of interest 206.

Next, the distal end of the an inner member may be inserted in the proximal end of the access member 208. The access member may comprise a discontinuity in the sideport cross-section and/or an internal structure to reduce the inner volume adjacent to the sideport. In an embodiment, the access member comprises a handle having a side aperture for insertion of the inner member. To expel the material from the distal end of the access member, the inner member is pushed through the access member 210.

Once the required amount of the material in the access member has been emplaced in the body part of interest, the used access member may be removed from the cannula. To remove the access member, the access member may be repositioned 212 to introduce a fracture or other point of discontinuity in the material being delivered. Once the access member is dislodged from the emplaced material, it may be removed from the subject 214. At this point, another access member may be loaded with additional material to be delivered 204, and positioned to have its distal end inserted in the body part of interest 206. The material in the second access member may then be delivered to the body part in a manner substantially as described for the first access member by repeating steps 204-214. Additional access members may be used until the correct amount of material is delivered to the body part of interest 216.

Method of Manufacture of Products, Systems and Kits

In other embodiments, the present invention may comprise a method of providing a product, system, or a kit for delivery of a material to a predetermined location in a subject. In certain embodiments the product, system, or a kit for delivery of a material to a predetermined location reduces the tendency of the delivery device to become embedded in the material that has been delivered to the body part.

In an embodiment, the method comprises producing a product comprising an access member, wherein the access member comprises a side port aperture positioned near the distal end of the access member for extruding the material to the body part, and wherein the access member comprises a variation in the internal diameter adjacent to the side port aperture. In one embodiment, the variation in the internal volume adjacent to the side port aperture reduces the tendency of the material extruded from the side port to adhere to the material remaining in the access member. Also, in certain embodiments, the products of the present invention reduce the amount of force needed to remove the access member from the material that has been delivered to the body part.

In another embodiment, the method may comprise providing a product for delivery of a material to a predetermined location in a subject, where the method comprises manufacturing a structure for use with an access member having a side port for extruding a material to a predetermined location within a subject, such that insertion of the structure into the access member introduces a variation in the internal diameter or internal volume adjacent to the side port aperture.

The access member may comprise a variety of means by which the access member comprises a variation in the internal diameter adjacent to the side port aperture, and wherein the variation in the internal volume adjacent to the side port aperture reduces the tendency of the material extruded from the side port to adhere to the material remaining in the access member and/or for the access member to become embedded in the material that has been delivered to the body part. In an embodiment, the access member is made to have a discontinuity in the internal volume of the access member adjacent to at least a portion of the side port. In certain embodiments, a structure is positioned in the access member to reduce the inner volume or diameter for a portion of the access member. For example, the discontinuity in the internal volume of the access member may comprise a structure positioned adjacent to the side port such that the structure reduces the internal volume of the access member at one end of the side port as compared to the other end of the side port. For example, in one embodiment, the structure reduces the diameter of the access member at the distal end of the side port such that the cross-section of the material as it exits the side port is smaller at the distal end than at the proximal end.

The structure for inserting into the access member and positioning adjacent to the side port aperture may comprise a variety of shapes. For example, in certain embodiments, the structure may comprise at least one of a straight ramp, a convex ramp, a compound convex ramp, a concave ramp, a compound concave ramp, a step, an angled step, a reverse angled step, a plurality of surfaces comprising at least one convex surface and one concave surface. The structure may also comprise, in alternate embodiments a plurality of steps, a wave or plurality of waves, or combinations thereof. Or, similar shapes may be used. For example, in an embodiment, the insertable structure may comprise an angled step, such that positioning the angled step adjacent to the side port reduces the internal volume of the access member at the distal end of the side port. Alternatively, the insertable structure may comprise at least one vertical step, such that positioning the at least one step adjacent to the side port reduces the internal volume of the access member at the distal end of the side port.

The structure that provides a variation in the internal diameter or cross-sectional volume of the access member adjacent to the side port may be fixedly attached to the access member so as to comprise a permanent part of the access member. Or, the structure that provides a variation in the internal diameter or cross-sectional volume of the access member adjacent to the side port may be removably attached to the access member.

In the embodiment where the structure is an accessory feature, the insertable structure may comprise an element to position the fixture adjacent to the side port. For example, the insertable structure may comprise an element to position the structure adjacent to the side port so that the structure has the correct alignment with respect to the length of the access member as well as being in the correct orientation to the side port opening. In the case of the ramp, the ramp may be positioned such that ramp extends from the wall that is opposite to the proximal end of the opening to the distal end of the opening (see e.g., FIG. 4). In an embodiment, the structure may comprise threads, such that the structure is threaded onto an access member having a removable cap. Or, the structure may comprise pins, or another type of element that may be used to align the structure (e.g., the ramp or steps) with the side port of the access member. Also, the structure may comprise an element to ensure that the structure is properly aligned along the length of the access member as described herein.

In an embodiment, the structure for use with an access member having a side port so as to introduce a discontinuity in the internal volume of the access member adjacent to the side port aperture may be solid. Or, the structure may be at least partially hollow. In an embodiment, the structure is constructed so as to be sufficiently rigid such it does not bend when inserted into the access member. The structure for use with an access member so as to introduce variation in the inner cross-sectional volume or inner diameter of the access member adjacent to the side port may be made of any material that is appropriate for use within a human or animal body. The structure may, in certain embodiments, be made of a material that is compatible with the other parts of the system. For example, the structure may be made of metal such as aluminum, stainless surgical steel, spring steel, a nickel titanium alloy or other alloys. Or, the structure may be made of plastic, such as polypropylene, polyethylene, polyethyleneteraphthalate (PET), TEFLON®, ionomer, polycarbonate or nylon. Or, the structure may be made of silicates or liquid crystal polymers. In certain embodiments, the structure may be coated with any appropriate commercially available medical grade coating including an anti-infective, an anti-coagulant, a release coating, and/or a slipping agent. One of ordinary skill in the art having the benefit of this disclosure would appreciate that other materials, including those that are well-known to one in the art, may be applied to configure the structure used to decrease adhesiveness or cohesiveness in the material being delivered to a body part described herein.

For example, an insertable element, such as an angled step, may be machined as a separate part (e.g., plug) to be inserted in the access member. The insertable part may comprise cylindrical distal portion that fits within the inner volume of the access member. In an embodiment, the insertable part may also comprise a distal end that can be substituted for the distal end of the access member. The insertable part may be manufactured to comprise a portion to allow the insertable part to by screwed onto the distal end of an access member that has a screw cap as the distal end.

The insertable part may comprise a sloped surface that ranges in slope from about 2 degree to 90 degrees, or from about 5 degrees to about 80 degrees, or from about 10 degrees to about 70 degrees, or from about 10 degrees to about 60 degrees, or from about 15 degrees to about 50 degrees, or from about 20 degrees to about 35 degrees, or from about 20 degrees to about 30 degrees. Or, ranges within these ranges may be used. Or a compound surface may be used such that slope may change along the length of the insertable part (i.e., the length of the access member). Or, steps or waves may be used. For example, embodiments of a compound surface, steps, wave and/or waves (or combination thereof) may have an overall slope that ranges from about 5 to 60 degrees, or from about 10 to 50 degrees, or from about 20 to 40 degrees, or is about 20 to 35 degrees, or 20 to 30 degrees. The surface of the ramp or other surface may be machined to be flat, concave, or convex as described herein. Also, the base of the insertable part may be tapered, or may be substantially squared off, to be flush with the internal circumference of the access member.

The insertable structure may be machined to fit snugly within the inner volume of an access member used to deliver material to a bone. For example, in alternate embodiments, the base of the ramp may range in length from about 0.1 to 0.6 inches, or from about 0.2 to about 0.5 inches, or from about 0.2 to about 0.45 inches, or may be about 0.3 to 0.4 inches. To fit within the distal end of a bone filler device, the distal portion of the insert (i.e., the portion of the insert excluding the ramp) may in alternate embodiments, range in length from about 0.04 to 0.15 inches, or from about 0.06 to about 0.12 inches, or from about 0.07 to about 0.1 inches, or may be about 0.08 to 009 inches. Or ranges within these ranges, or other ranges described herein may be used.

Also, in alternate embodiments, the width (or diameter) of the insertable part (e.g., such as plug 52) is such that when inserted in the access member, there is minimal or no detectable space between the inner wall of the access member and the outer wall of the insertable part, such that the insertable piece fits snugly in the access member. Thus, for a bone filler device, the width or outer diameter (OD) of the insertable part may range from about 0.135 to about 0.085 inches, to about 0.125 to about 0.095 inches, to about 0.115 to about 0.105 inches, or may be about 0.111 inches. Where the insertable part includes a distal end that may replace the end of the access member, the insertable piece may be sized to be the same as the end of the access member. Thus, in alternate embodiments, the width or outer diameter (OD) of the distal end of the insertable part may range from about 0.165 inches to about 0.109 inches, or from about 0.140 inches to about 0.120 inches, or from about 0.135 inches to about 0.130 inches, or may be about 0.134 inches. Or ranges within these ranges, or other ranges described herein may be used.

As discussed above, the distal end of the insertable piece may be threaded to allow for the part to form the distal end of the access member. Also, the threads (or pins or other alignment device) may provide a means by which the insertable part is properly aligned with the side port of the access member.

In yet another embodiment, the shape of the access member provides a means to decrease adherence of the material being delivered to a body part to the material remaining in the access member or to the access member itself. Thus, in certain embodiments, the access member may be made so as to comprise a reduction in the external cross-section of at least a portion of the distal end of the access member that includes the side port aperture. In an embodiment, the access member is fashioned to comprise a radius that exhibits a reduction in size along the length of the access member, such that the access member has a smaller radius at the distal end than at the proximal end (see e.g., FIG. 9A). In an embodiment, both the internal radius, and the external radius decrease from the proximal end to the distal end of the access member. Or, the access member may be tapered at the distal end, but not throughout the entire length. For example, in an embodiment, the access member is tapering of the access member begins at about the point where the side port aperture is positioned, or just proximal (e.g., about 1 inch, 0.5 inch, 0.3 inch proximal) thereto. Thus, in various alternate embodiments, the access member may be made to comprise an internal radius and/or external radius that are significantly narrowed along the length of, and/or adjacent to the opening.

The external surface of the access member may be curved so as to aid in removal of the access member from the material deposited in the body part. For example, the access member may be made to comprise a concave outer surface that is apposite the side port aperture. In certain embodiments, the outer surface may mimic a concave curvature of inner surface of the access member that is adjacent to the side port aperture. In an embodiment, the access member may be fashioned such that the curved outer surface extends to a narrow tip at the end of the access member thereby reducing the tendency of the access member to become embedded in the material that has been extruded into the body part. Or, the access member may be made so as to comprise an indentation along the outer surface which provides either a flat back side to the aperture and/or a convex curvature to the internal radius of the access member as described herein. In this way, a material will be directed out of the access member and away from the back side of the opening.

In certain embodiments, the access member may be coated with a material to help reduce adhesion of the access member to the material being extruded from the access member. In an embodiment, at least a portion of the distal end of the access member that includes the side port aperture is coated with a plastic, such as TEFLON® or the like.

In an embodiment, the portion of the access member that provides a variation in the internal volume adjacent to the side port aperture may be coated with a substance that has the ability to shrink and expand. The coating substance may then provide a cushion, such that if a material being delivered to a body part hardens, the cushioning substance will absorb most of the shrinkage induced by the cured material. In an embodiment, the coating substance has a low frictional coefficient (i.e., is slippery). In an embodiment, the coating substance is made of TEFLON® (e.g., PFA from Dupont), silicon, polyethylene, polyurethane, and the like. In certain embodiments, the portion of the access member that provides a variation in the internal volume adjacent to the side port aperture may be coated with any appropriate medical grade coating including an anti-infective, an anti-coagulant, a release coating, and/or a slipping agent such as those described herein.

As described above, the side port aperture may be fashioned to provide a means to induce reduced adherence of the material being delivered to a body part to either the access member or any material remaining within the access member. In certain embodiments, the side port aperture may be shaped such that upon delivery of at least a portion of the material contained within the access member to the body part via the side port aperture, the shape of the side port aperture reduces the tendency of the material extruded from the side port to adhere to the material remaining in the access member. Thus, in certain embodiments, the side port aperture is shaped such that the adhesive material in the body part displays less adhesion to the access member and any material remaining within the access member upon retraction of the access member from the body part as compared to when a round or oval side port aperture is used. For example, the side port may be shaped such that a discontinuity is introduced into the material at certain points as the material exits the access member. In one embodiment, the side port may be shaped such that the material is sheared at least in part at certain points as it exits the access member. Or, the side port may be shaped such that a fracture point is induced in the material at certain points as the material exits the access member. Or, the side port may be shaped such that there is a point at which the material is forced through an aperture that comprises series of points or edges. In this way, the material may easily fracture as the access member is twisted away from the extruded or emplaced material. Thus in certain embodiments, the access member may be shaped so as to comprise a discontinuity in the shape of the side port. In certain embodiments, the side port may comprise at least one of a serrated edge, a discontinuity in the shape of aperture such that the width of at least one portion of the aperture is substantially reduced in size as compared to the width at another portion of the aperture, or a division of the aperture into a plurality of apertures. Alternatively or additionally, there may be a portion of the side port that comprises a plurality of serrated or straight edges. For example, the access member may be fashioned so as to comprise a side port shaped as a compound curve, or a tear drop, or a diamond, or two (or more) interlaced diamonds, or an oval with serrated edges, or an eye, or a bifurcated side port, or serrated bifurcated side port, may be used.

As described herein, the access member may provide a path to access a region or a body part that is located within a subject's body. The access member may be any type of device that can extend from the location of interest (e.g., a bone or an organ) to be accessible to an individual accessing the location of interest. For example, the access member may be designed to extend from an internal body part in a subject to outside of the subject's body. In various embodiments, the access member may be substantially cylindrical in shape. Or, the access member and segments may be other shapes, such as oval, rectangular, polygonal (e.g., hexagonal, octagonal) and the like. The access member may comprise an elongated hollow member such as a hollow cylinder or a tube. For example, the access member may comprise a cannula, such as a cannula used to deliver a material to bone or another type of body part.

The access member may be constructed, for example, using standard, flexible, medical grade plastic materials, such as those described herein. Example materials include, but are not limited to vinyl, ionomer, polypropylene, polyethylene, PET, or nylon. In some embodiments, the access member may comprise a metal. Thus, in alternate embodiments, the access member, like other parts of the system, may comprise aluminum, stainless steel, spring steel, nickel titanium, or other metal alloys. Sizes for the access member may depend on the body location being access. Thus, in alternate embodiments, where the product comprises an access member for a bone filler device, an access member may comprise dimensions on the order of about 2 to 30 inches, or from about 2 to 20 inches (50.8 mm to 508 mm), or about 4-15 inches (101 mm to 381 mm), or about 6 to 12 inches (152 mm to 305 mm), or about 8 inches (203 mm) in length. Also, in alternate embodiments, the outer diameter of the access member may range from about 0.04 to about 2 inches, or from about 0.04 to 0.8 inches, 0.05 to 0.5 inches (1.27 mm to 12.7 mm), or from about 0.1 to 0.4 inches (2.54 to 1.02) or from about 0.120-0.160 inches (3.05 mm to 4.1 mm), or may be about 0.134 inches (0.34) in diameter. Similarly, the inner diameter (ID) may, in alternate embodiments, range from about 0.02 to about 1 inch, or from about 0.07 to about 0.3 inches, or from about 0.1 to about 0.012 inches, or may be about 0.106 inches. For a tapered access member, the outer diameter may ranged from about 0.07 to 0.3 inches, or from about 0.150 to 0.125 inches at the distal end, to 0.125 to 0.100 at the portion of the access member just proximal to the opening, then tapering off to a point distal to the opening for a access member about 8-12 inches long. Or, ranges within these ranges may be used.

The method may further include the step of manufacturing an inner member. As described herein, the inner member may comprise a material that is flexible, such that the inner member is able to bend, or the inner member may comprise a material that is substantially rigid. Also, in an embodiment, the inner member may comprise a material that comprises shape memory, such as nitinol. Thus, in alternate embodiments, the inner member may be made of aluminum, stainless steel, spring steel, nickel titanium alloys, or other alloys. Or, in some embodiments, the inner member may be made of plastic. For example, a resilient plastic such as vinyl, nylon, polypropylene, a polyethylene, ionomer, polyurethane, and polyethylene tetraphthalate (PET) may be used.

In yet another embodiment, the method may comprise manufacturing an access member or a part thereof, or a fixture for an access member, comprising a side aperture for insertion of an inner member such as a rod, plunger, or other tamping instrument into the access member, so as to reduce the clearance required for insertion of the inner member into the access member when the access member is positioned in a subject.

In one embodiment, an end piece may be fashioned to include an injection side port to the access member. In one embodiment, the end piece may comprise a handle. The end piece may shaped to comprise an opening or a recessed portion that can engage the proximal end of the access member. The end piece may be made of a material that is compatible with the other parts of the system. For example, the end piece may be made of metal such as aluminum, stainless steel, stainless surgical steel, spring steel, a nickel titanium alloy or other alloys. Or, the end piece may be made of plastic, such as polypropylene, polyethylene, polyethyleneteraphthalate (PET), TEFLON®, ionomer, polycarbonate or nylon. Or, the end piece may be made of silicates or liquid crystal polymers. In one embodiment, the end piece may be fashioned as a handle molded from plastic.

The end piece having a side aperture may be shaped to attach to an access member and be used where there is limited clearance. For example, in alternate embodiments, an end piece fashioned as a handle may comprise a height (i.e., the dimension parallel to the axis of the access member) ranging from about 0.5 to 5 inches, or from about 0.75 to about 4 inches, or about 0.8 to 2 inches. The handle may comprise a width or diameter (i.e, perpendicular to the axis of the access member) that allows for the angle of entry of the inner member to be varied from a direct linear entry. In alternate embodiments, a handle comprising a side port may comprise a width in the ranging of from 1 to 5 inches, or from about 1 to about 4 inches, or about 1 to 3 inches. Or ranges within these ranges may be used.

Each of the components used in the products, systems, and kits of the present invention may comprise a material that may be sterilized by either chemical treatment, high temperature, and/or high pressure, exposure to sterilizing gas, or a combination of sterilization treatments as is known in the art. Also, the components of the products, systems, and kits of the present invention may be disposable, or may be formulated to allow for cleaning, re-sterilization, and re-use.

Embodiments of the present invention may provide certain advantages. For example, in an embodiment, using an access member having a means to reduce the tendency for the device to become embedded in the therapeutic material once the material hardens in situ. Also, using an access member having a means to induce a point of discontinuity in the material being delivered should allow for a small point of discontinuity to be formed such that when the device is extricated from the subject, any breaks in the material being delivered will be relatively minor.

Also, using an access member of the present invention may provide access to a body part where the clearance is limited. As described above, emplacement of a material at a location of interest in a subject, such as emplacing a bone filler material in bone, may require monitoring by fluorography, which in turn can require positioning the arm of an X-ray machine close to the subject's torso. Using an access member having a side port for insertion of the inner member in a bone filler device may allow for a physician to emplace bone filler material where clearance to insert a straight rod-like plunger is restricted due to the positioning of X-ray equipment, or for other reasons.

### EXAMPLES

### Example 1

A variety of cannulas having a reduced inner diameter adjacent to the side port have been manufactured. One cannula having a straight ramp positioned adjacent to a side port was made by forming an insert comprising a ramp, and inserting the ramp adjacent to the side port aperture.

The cannula was a stainless steel cylindrical cannula measuring 8 inches in length and having an internal diameter (ID) of 0.106 inches and an external diameter of 0.134 inches. The cannula was fashioned to have an oval side port aperture measuring about 0.08 inches in width by about 0.2 inches in length. The cannula was made to be open at the distal end. To form the insert, a solid stainless steel cylinder was formed having an outer diameter of about 0.106 inches along most of the length of the cylinder except at one end, which had an outer diameter of about 0.134 inches to form a cap-like structure at the distal end of the insert. The proximal end of the cylinder was formed into a ramp by milling the end of the cylinder. Once the insert with a ramp was formed, the piece was inserted into the distal end of the cannula and the ramp positioned opposite to the side port by welding the distal end of the insert (i.e., the cap-like structure) to the distal end of the cannula. Proper positioning of the ramp along the length of the cannula was due to the fact that the distal end of the insert limited how far the ramp could be inserted into the cannula.

### Example 2

A cannula having a tapered end was made by taking a standard 8 inch cannula (in this case the cannula had an OD of about 0.134 inches and an inner diameter of about 0.109 inches). The material was machined from OD of the distal end slowly removing less material going towards the proximal end, but not removing material from about 1.5 inch from the distal end. The distal end of the cannula was then roughened (up to about 1.5 inches from the distal end) to form small ridges in the surface. A plastic hollow cylindrical piece having the same OD as the metal cannula and an inner diameter of less than 0.109 inches was then molded to form a tapered end having a convex curvature for both the inner cross-section and the outer cross-section of the cylinder. The curvature was such as to provide an angle of about 30 degrees over the entire curvature. The roughened end of the metal cannula was then inserted into the proximal end of the distal piece, to form a cannula having a side port opening with a convex ramp and an concave outer distal end, similar to the cannula of FIG. 9A. A handle was attached to the proximal end.

It will be understood that each of the elements described above, or two or more together, may also find utility in applications differing from the types described. While the invention has been illustrated and described as devices, systems, kits and methods to deliver a material to an internal body part, it is not intended to be limited to the details shown, since various modifications and substitutions can be made without departing in any way from the spirit of the present invention. Where method and steps describe above indicate certain events occurring in certain order, those of ordinary skill in the art having the benefit of this disclosure would recognize that the ordering of certain steps may be modified and that such modifications are in accordance with the variations of the invention. Additionally, certain of the steps may be performed concurrently in a parallel process when possible, as well as performed sequentially as described above. As such, further modifications and equivalents of the invention herein disclosed may occur to persons skilled in the art using no more than routine experimentation, and all such modifications and equivalents are believed to be within the spirit and scope of the invention as described herein. All patents and published patent applications referred to in this document are incorporated by reference in their entireties as if each individual publication or patent application were specifically and individually put forth herein.

That which is claimed is:

1. A method for delivery of a material to a body part in a subject using an access member configured to provide percutaneous surgical access to the body part comprising the steps of:
inserting the access member in the subject such that the distal end of the access member is positioned within the body part, or juxtaposed adjacent to an aperture in the body part, wherein at least a portion of the internal volume of the access member comprises at least a portion of the material to be delivered to the body part;
urging an inner member at least partially through the access member to deliver at least a portion of the material to the body part, wherein the access member comprises a side port aperture positioned near the distal end of the access member for extruding the material to the body part, and wherein the access member comprises a variation in the internal diameter adjacent to the side port aperture, and wherein the variation in the internal volume adjacent to the side port aperture reduces the tendency of the material extruded from the side port to adhere to the material remaining in the access member.

2. The method of claim 1, wherein the variation in the internal diameter adjacent to the side port aperture comprises a reduction in the internal diameter at the distal end of the side port aperture as compared to the proximal end of the side port aperture.

3. The method of claim 1, wherein the material for delivery to the body part comprises a bone filler or bone cement.

4. The method of claim 1, wherein the variation in the internal diameter adjacent to the side port aperture is provided by a structure positioned within the internal volume of the access member such that at least a portion of the structure is adjacent to the side port aperture.

5. The method of claim 4, wherein the structure used to provide variation in the internal diameter adjacent to the side port aperture comprises at least one of a straight ramp, a convex ramp, a compound convex ramp, a concave ramp, a compound concave ramp, a step, an angled step, a reverse angled step, or a plurality of surfaces comprising at least one convex surface and one concave surface.

6. The method of claim 1, wherein the access member further comprises a reduction in the external cross-section of at least a portion of the distal end of the access member that includes the side port aperture.

7. The method of claim 1, wherein at least a portion of the distal end of the access member that includes the side port aperture is coated with a plastic.

8. The method of claim 1, wherein the side port aperture comprises a shape such that upon delivery of at least a portion of the material contained within the access member to the body part via the side port aperture, the shape of the side port aperture reduces the tendency of the material extruded from the side port to adhere to the material remaining in the access member.

9. The method of claim 1, wherein the side port comprises at least one of a serrated edge, a discontinuity in the shape of the aperture such that the width of at least one portion of the aperture is substantially reduced in size as compared to the width of another portion of the aperture, or a division of the aperture into a plurality of apertures.

10. The method of claim 1, wherein the body part comprises at least one of a vetebral body or a spinal disc.

11. A product comprising an access member configured to provide percutaneous surgical access to a body part in a subject comprising:
a distal end for insertion into a body part and a proximal end for access by a user; and
a side port aperture positioned near the distal end of the access member for extruding a material to a body part within a subject, wherein the access member comprises a variation in the internal diameter adjacent to the side port aperture, and wherein the variation in the internal volume adjacent to the side port aperture reduces the tendency of the material extruded from the side port to adhere to the material remaining in the access member.

12. The product of claim 11, wherein the material for delivery to the body part comprises a bone filler or bone cement.

13. The product of claim 11, wherein the variation in the internal diameter adjacent to the side port aperture comprises a reduction in the internal diameter at the distal end of the side port aperture as compared to the proximal end of the side port aperture.

14. The product of claim 11, wherein the variation in the internal diameter adjacent to the side port aperture is provided by a structure positioned within the internal volume of the access member such that at least a portion of the structure is adjacent to the side port aperture.

15. The product of claim 14, wherein the structure is removably attached to the access member.

16. The product of claim 11, wherein the variation in the internal diameter adjacent to the side port aperture comprises at least one of a straight ramp, a convex ramp, a compound convex ramp, a concave ramp, a compound concave ramp, a step, an angled step, a reverse angled step, or a plurality of surfaces comprising at least one convex surface and one concave surface.

17. The product of claim 11, wherein the access member further comprises a reduction in the external cross-section of at least a portion of the distal end of the access member that includes the side port aperture.

18. The product of claim 11, wherein the side port aperture comprises a shape such that upon delivery of at least a portion of the material contained within the access member to the body part via the side port aperture, the shape of the side port aperture reduces the tendency of the material extruded from the side port to adhere to the material remaining in the access member.

19. The product of claim 18, wherein the side port comprises at least one of a serrated edge, a discontinuity in the shape of aperture such that the width of at least one portion of the aperture is substantially reduced in size as compared to the width of another portion of the aperture, or a division of the aperture into a plurality of apertures.

20. A product comprising an insert to be emplaced in the internal volume of an access member, wherein the access member is configured to provide percutaneous surgical access to a body part in a subject, and wherein the access member comprises a distal end for insertion into a body part and a proximal end for access by a user and a side port aperture positioned near the distal end of the access member for extruding a material to a body part within the subject, such that insertion of the structure into the access member provides a variation in the internal diameter of the access member adjacent to the side port aperture.

21. The product of claim 20, wherein the variation in the internal diameter adjacent to the side port aperture comprises a reduction in the internal diameter at the distal end of the side port aperture as compared to the proximal end of the side port aperture.

22. The product of claim 20, wherein the variation in the internal diameter adjacent to the side port aperture comprises at least one of a straight ramp, a convex ramp, a compound convex ramp, a concave ramp, a compound concave ramp, a step, an angled step, or a plurality of surfaces comprising at least one convex surface and one concave surface.

23. A handle for an access member configured to provide percutaneous surgical access to the body part, wherein the access member comprises a distal end for insertion into a body part and a proximal end for access by a user, and wherein the handle comprises an aperture for insertion of an inner member into the distal end of the access member such that the inner member utilizes a curvilinear path as it travels through at least a portion of the handle.

24. The handle of claim 23, wherein the aperture for insertion of the inner member is positioned so as to reduce clearance for the inner member at the proximal end of the access member during insertion of the inner member into the access member when the access member is positioned in a subject.

25. A method comprising the steps of:
(a) positioning an access member comprising a side port aperture for emplacing a material in a body part, wherein the access member comprises a material to be emplaced in the body part;
(b) delivering at least a portion of the material to the body part via the side port aperture;
(c) intentionally leaving the access member in the body part until the material has hardened or cured; and
(d) removing or repositioning the access member in a manner such that the side port is used to sever material that remains in the distal portion of the access member from material that has been emplaced in the body part.
